(19) United States
(12) Patent Application Publication
(10) Pub. No.: US 2024/0325728 A1
(43) Pub. Date: Oct. 3, 2024

(54) ELECTRODES FOR NEUROMODULATION

(71) Applicant: Cala Health, Inc., San Mateo, CA (US)

(72) Inventors: Gregory T. Schulte, Oakland, CA (US); Alexander R. Kent, Mountain View, CA (US); Jessica M. Liberatore, San Mateo, CA (US); John Vincent Colombo, Burlingame, CA (US)

(21) Appl. No.: 18/294,496

(22) PCT Filed: Jul. 19, 2022
(86) PCT No.: PCT/US2022/037600

§ 371 Date: Feb. 1, 2024

Related U.S. Application Data
(60) Provisional application No. 63/203,894, filed on Aug. 3, 2021.

(51) Int. Cl. A61N 1/04; A61N 1/36

(52) U.S. Cl. CPC A61N 1/0496; A61N 1/0456; A61N 1/36003; A61N 1/36021; A61N 1/36031

(57) ABSTRACT

Disclosed herein are systems, devices, and methods for peripheral nerve stimulation. A wearable neuromodulation (e.g., neurostimulation) device can accomplish target nerve stimulation using circumferential electrode configuration, in-line electrode configuration, or concentric electrode configuration. In some embodiments, the electrodes may obtain a self-wetting capability by comprising an antistatic material, which may bloom to the surface of the electrode and serve as a layer of conductive lubricant to improve electrical conductivity between the electrode and skin. In some embodiments, the device may deliver stimulation through the electrodes to induce sweat and the induced sweat may serve as a wetting agent to improve electrical conductivity. In some embodiments, reservoirs capture a user's sweat or hold other wetting agents filled by the user, and the captured sweat and/or other wetting agents may be delivered to the area between the electrode and skin by using capillary action, osmosis, or micropump.

### REFERENCE TO RELATED APPLICATIONS

This application claims the benefit of U.S. Provisional Application No. 63/203,894, filed Aug. 3, 2021, which is hereby incorporated by reference in its entirety.

### BACKGROUND

### Field

Some embodiments of the invention relate generally to systems, devices, methods for neuromodulating (such as stimulating) nerves, and methods of manufacture, and more specifically relate to wearable devices that include a band or other wearable for releasably securing a stimulator on a user's limb or other body part for enhancing electrically stimulating peripheral nerve(s) with one or more electrodes to treat various diseases and disorders.

### Description of the Related Art

A wide variety of modalities can be utilized to neuromodulate peripheral nerves. For example, Applicant's own work has demonstrated that electrical energy can be delivered transcutaneously via electrodes on the skin surface with neurostimulation systems to stimulate peripheral nerves, such as the median, radial, and/or ulnar nerves in the upper extremities; the tibial, saphenous, and/or peroneal nerve in the lower extremities; or the auricular vagus, auriculotemporal, trigeminal or cranial nerves on the head or ear, as non-limiting examples. A number of conditions, such as tremors, can be treated through some form of transcutaneous, percutaneous, or other implanted forms of peripheral nerve stimulation.

### SUMMARY

Wearable systems to neuromodulate nerves with compact, ergonomic form factors are needed to enhance efficacy, compliance, and comfort with using the devices. The wearable system can include a stimulator and detachable band. The stimulator can be attached to a band that is wrapped around a patient's wrist or other body part. Ear (auricular) embodiments are also provided. The band is worn throughout the day including during daily activities.

The device can include two or more effectors, e.g. electrodes, for providing neurostimulation signals to the patient. The electrodes of the device can employ a wetting agent in some situations (water, gel for example) to ensure a low resistance connection between the electrode and the patient's skin. However, as the length of therapy is increased or the length of wear time is increased, the patient may have to rewet their skin to ensure that adequate electrical conductivity between the electrodes and the patient's skin is maintained. Maintaining adequate electrical conductivity allows the device to comfortably deliver electrical current during a stimulation session. However, in some cases, water can evaporate over the course of a stimulation session which can potentially increase the risk of patient discomfort or skin irritation.

Disclosed herein are systems and methods which can induce the patient to sweat at the location of the electrode. In this way, sufficient electrode-skin conductivity is maintained during the stimulation session. The induced sweat can be more conductive than water and naturally accumulate at the electrode-skin interface. The amplitude of the stimulation signal can be selected to activate C-type sudomotor neurons in a target nerve(s) that innervate downstream (anterograde) sweat glands to increase the degree of sweating. If alternative stimulation parameters are needed to deliver a main therapy, then the systems and methods may periodically alternate between delivering stimulation with parameters for this main therapy and sweat inducing parameters.

The systems and methods can reduce patient discomfort (if caused by the sweat inducing parameters) by employing, for example, a bipolar electrode configuration to activate Aδ and C-type pain fibers of the patient to block the resultant afferent pain signals caused by the sweat inducing parameters.

Another modality for inducing sweat is the placement of a resistive heating element and/or thermoelectric cooling element on the patient's skin. The resistance heating element creates a temperature differential which naturally induces sweating.

Another modality for ensuring a low resistance connection between the electrode and the patient's skin is to employ a self-wetting material in the device. The self-wetting material can be incorporated into material of the electrode. For example, an antistatic material can be added to the electrode material that reacts with moisture to create conductive pathways. The antistatic material may be incompatible with silicone electrodes which advantageously causes the antistatic material to bloom to the surface of silicone electrode when exposed to the warm skin of the patient. The warm skin causes the antistatic material to naturally bloom to the skin surface creating a non-sticky, moisturizing, conductive lubricant. The antistatic material can be mixed with the silicone during manufacture of the electrode without adversely impacting the material properties of the silicone. The amount of bloom of the antistatic material can be self-limiting in that blooming stops once a film of the antistatic material has bloomed on the electrode.

The systems and methods disclosed herein can manufacture the silicone material to have a desired hardness. The level of hardness can be selected depending on the expected body location for the device as well as the planned wear time of the device. For example, a patient may prefer a softer silicone material for the electrode when the device is worn for longer periods of time even though the softer material may adversely impact the durability of the electrode. A patient may prefer a harder silicone material when worn for shorter periods of time or on a body location that is less susceptible to discomfort.

Another modality embodiment for ensuring a low resistance connection between the electrode and the patient's skin is to employ a reservoir or chamber of wetting agent in the device. The reservoir or chamber may (1) include channels from which a patient's own sweat has been intentionally captured; (2) be pre-filled or patient-refilled to store water/electrolyte fluid/gel. The wetting agent (e.g., sweat/water/electrolyte fluid/gel) can be slowly transferred from the reservoir or chamber to a location between the patient's skin and the surface of the electrode. The slow transfer of the wetting agent may be performed using capillary action, osmosis, micropump, or other means.

The transfer of the wetting agent may be passive or it may be intentionally activated by the patient. A passive transfer can employ microchannels, grooves, or ridges that are molded into the electrodes and/or device that serve as sweat reservoirs or chambers. A patient can trigger an active transfer by, for example, electrical or mechanical means. For example, the patient can electrically activate the transfer or mechanically activate the transfer to cause the device to cause the wetting agent to flow into the area between the surface of the electrode and patient's skin. Active and passive triggering can be employed either independently or in combination to help retain moisture and sweat that lowers surface capacitance.

The electrodes employed by some systems and methods are located on the device so as to contact multiple locations along an outer circumference of the patient's wrist. However, certain locations are more susceptible to intermittently disconnecting from the patient's skin during normal patient activity throughout the day. These intermittent disconnections can cause variations in the electrical conductivity between the electrode and the patient's skin. Such variations can result in patient discomfort in some cases.

Systems and methods disclosed herein can, for example, reduce or avoid such patient discomfort by placing the electrodes at locations that reduce the likelihood that intermittent disconnections may occur. For example, a concentric electrode configuration as well as an inline modified electrode configuration are disclosed herein. These configurations can constrain the spread of electrical current during nerve stimulation, which may reduce neural activation thresholds, and decrease the likelihood for discomfort occurring even if an electrode intermittently disconnects from the skin.

In some embodiments, a self-wetting electrode for transcutaneous electrical stimulation is provided. The self-wetting electrode, for example, comprises, consists, or consists essentially of a conductive backing layer and a skin contact layer. The skin contact layer is disposed on the conductive backing layer and is configured to deliver electrical current from the conductive backing layer to the skin for transcutaneous electrical stimulation. The skin contact layer may comprise an antistatic material configured to bloom to a skin facing surface of the skin contact layer to form a layer of conductive lubricant when the skin contact layer is in contact with a heat source. The heat source may be body heat. The antistatic material may bloom to the skin facing surface of the skin contact layer to form a layer of conductive lubricant when the self-wetting electrode is placed on a desired location of a user's skin. The antistatic material may stop blooming to the skin facing surface after the layer of conductive lubricant covers substantially all the area of the skin facing surface. The conductive lubricant may be non-sticky and moisturizing. The layer of conductive lubricant may provide a higher electrical conductivity between the skin contact layer and a skin location where the self-wetting electrode is placed on comparing to the electrical conductivity between the skin contact layer and the skin location without the layer of conductive lubricant.

In some embodiments, a method of delivering transcutaneous electrical stimulation to a user is provided. The method, for example, comprises, consists, or consists essentially of: providing a wearable device comprising at least 2 self-wetting electrodes comprising a conductive backing layer, and a skin contact layer comprising an antistatic material configured to bloom to a skin facing surface of the skin contact layer to form a layer of conductive lubricant when the skin contact layer is in contact with the user's skin; positioning the skin contact layer of the self-wetting electrodes on desired locations on the user's skin, wherein the skin facing surface of the skin contact layer is in direct contact with the skin; waiting for the antistatic material to bloom to the skin facing surface of the skin contact layer and covers substantially all the area of the skin facing surface; and activating the device, thereby delivering electrical current through the self-wetting electrodes to the desired locations on the skin.

In some embodiments, a wearable device for delivering transcutaneous electrical stimulation to a user is provided. The wearable device, for example, comprises, consists, or consists essentially of a wearable band comprising a strap configured to be worn around a body part; at least two electrodes disposed on the strap; a flexible circuit disposed within the strap; a controller; a power source; and at least one reservoir configured to store wetting agent. The at least two electrodes each may comprise a conductive backing layer and a skin contact layer disposed on the conductive backing layer and the skin contact layer may be configured to deliver electrical current from the conductive backing layer to the skin for transcutaneous electrical stimulation. The at least one reservoir may be in the band and/or electrodes, and the flexible circuit may be in electrical communication with the at least two dry electrodes and the controller.

In some embodiments, a method of wetting an electrode for transcutaneous electrical stimulation is provided. The method comprises, consists, or consists essentially of: providing a wearable device comprising at least 2 electrodes comprising a conductive backing layer and a skin contact layer comprising a skin facing surface; positioning the electrodes on desired locations on a user's skin, wherein the skin facing surface of the skin contact layer is in direct contact with the skin; and activating the device to perform a sweat induction session with a first electrical stimulus and a tremor treatment session with a second electrical stimulus after the sweat induction session through the electrodes on the desired locations of the user's skin. The first electrical stimulus may be configured to induce sweat between the skin facing surface of the skin contact layer and the user's skin and the second electrical stimulus may be configured to deliver the transcutaneous electrical stimulation to treat tremor.

In some embodiments, a method of delivering transcutaneous electrical stimulation to a user is provided. The method comprises, consists, or consists essentially of: providing a wearable device comprising at least one self-wetting electrode comprising a conductive backing layer, and a skin contact layer comprising an antistatic material configured to bloom to a skin facing surface of the skin contact layer to form a layer of conductive lubricant when the skin contact layer is in contact with skin of the user; positioning the skin contact layer of the at least one self-wetting electrode on a desired location on the skin, wherein the skin facing surface of the skin contact layer is in direct contact with the skin; determine an impedance of the self-wetting electrode; and activating the wearable device at least in part based on the impedance, thereby delivering electrical current through the at least one self-wetting electrode to the desired location on the skin. In some embodiments, the impedance is based at least in part on a relative conductance between the peripheral nerve electrode and the skin of the user.

In some embodiments, an in-line electrode system for delivering transcutaneous electrical stimulation to a target nerve is provided. The in-line electrode system comprises two parallel counter electrodes and a stimulating electrode between and parallel to the two parallel counter electrodes. The stimulating electrode and the two counter electrodes are configured, for example, to be placed longitudinally along the target nerve on the same side of the arm relative to the target nerve.

In some embodiments, a concentric electrode system for delivering transcutaneous electrical stimulation to a target nerve is provided. The concentric electrode system, for example, comprises a stimulating electrode and a counter electrode, wherein the stimulating electrode is configured to be placed around the counter electrode.

In some embodiments, any of the devices or methods are used for treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease).

In several embodiments, one or more of bradykinesia, dyskinesia, gait dysfunction, dystonia and/or rigidity are treated with the devices and methods described herein (e.g., in connection with Parkinson's disease or in connection with other disorders). Rehabilitation of movement is treated in some embodiments (for example to restore or improve movement and motion) in subjects who have suffered from an acute or chronic event including, for example, cardiac events (such as atrial fibrillation, hypertension, and stroke, inflammation, neuroinflammation, etc.). Epilepsy is treated in one embodiment. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation), headache, back pain, joint pain and stiffness, muscle pain and tension, etc.

### BRIEF DESCRIPTION OF THE DRAWINGS

The following drawings are for illustrative purposes only and show non-limiting embodiments. Features from different figures may be combined in several embodiments.

FIG. 1A illustrates a block diagram of an example neuromodulation (e.g., neurostimulation) device.

FIG. 1B illustrates a block diagram of an embodiment of a controller that can be implemented with the hardware components described with respect to FIG. 1A.

FIG. 1C schematically illustrates a neurostimulation device and base station.

FIGS. 2A-2C illustrate various embodiments of electrodes on a wrist, including a charge-balance electrode on the back of the wrist to reduce the number of electrodes needed to stimulate multiple nerves and electrodes positioned on the circumference of the wrist to selectively stimulate the nerves target for excitation.

FIG. 3 illustrates an example embodiment of a concentric electrode system to deliver nerve stimulation.

FIGS. 4A-4C illustrate various example embodiments of an in-line electrode system to deliver nerve stimulation.

FIGS. 5A-5I illustrate various charge balanced waveforms and schematics of electrode configurations that can be used to stimulate a nerve.

FIGS. 6 and 7 illustrate various example embodiments of a wearable band with two electrodes.

FIGS. 8A-8H illustrate various example embodiments of dry electrodes.

FIGS. 9A-9C illustrate views of mechanical elements, such as holes, that can allow the skin contact layer to surround the base layer and maintain good contact.

FIG. 9D illustrates a star-shaped dry electrode, according to some embodiments of the invention.

FIG. 9E illustrates schematically a cross-section of a dry electrode with a variable filler concentration.

FIGS. 10A-10B illustrate views of electrode geometries with non-sharp edges and corners.

FIGS. 11A-11B schematically illustrate an example embodiment of an electrode before and after the antistatic material blooms to the surface of the electrode.

FIG. 12A schematically illustrates the direction of the stimulation to induce sweat according to some embodiments.

FIG. 12B illustrates an example electrical current waveform to induce sweat according to some embodiments.

### DETAILED DESCRIPTION

Disclosed herein are devices configured for providing neuromodulation (e.g., neurostimulation). The neuromodulation (e.g., neurostimulation) devices provided herein may be configured to stimulate peripheral nerves of a user. The devices may be configured to be coupled to the surface of a user's skin for transcutaneous stimulation via two or more effectors, e.g. electrodes. The devices can comprise any combination of features disclosed in any of the figures. Accordingly, the devices can have any number of different configurations. Thus, while certain combinations of features are illustrated in each figure, the features are not limited to only being incorporated as part of the illustrated combinations. In this way, any of the features disclosed in any of the figures can be employed with any other feature disclosed in any of the figures. For simplicity of description, certain combinations of features were selected to be illustrated in any given figure. However, the selected combinations of features do not limit the disclosure.

The neuromodulation (e.g., neurostimulation) devices may be configured to transmit one or more neuromodulation (e.g., neurostimulation) signals across the skin of the user. In many embodiments, the devices are wearable devices configured to be worn by a user. The user may be a human, another mammal, or other animal user. The system could also include signal processing systems and methods for enhancing diagnostic and therapeutic protocols relating to the same. In some embodiments, the device is configured to be wearable on an upper extremity of a user (e.g., a wrist, forearm, arm, and/or finger(s) of a user). In some embodiments, the device is configured to be wearable on a lower extremity (e.g., ankle, calf, knee, thigh, foot, and/or toes) of a user. In some embodiments, the device is configured to be wearable on the head or neck (e.g., forehead, ear, neck, nose, and/or tongue). Single or multiple bands that partially or fully encircle a limb (such as a wrist, ankle, arm, leg) are provided in some embodiments. Ear devices are also provided in some embodiments that can be used with or without a limb band. In one embodiment, an ear device and a wrist band are provided for synergistic treatment.

In several embodiments, dampening or blocking of nerve impulses and/or neurotransmitters are provided. In some embodiments, nerve impulses and/or neurotransmitters are enhanced.

In some embodiments, the device is configured to be wearable on or proximate an ear of a user, including but not limited to auricular neuromodulation (e.g., neurostimulation) of the auricular branch of the vagus nerve, for example. One or more of the vagus, auriculotemporal, great auricular, trigeminal or cranial nerves may be treated in some embodiments. In some embodiments, only the vagus nerve is neuromodulated. In some embodiments, the vagus nerve and one, two or more other nerves are neuromodulated (e.g., trigeminal nerve, great auricular nerve, nerves of the auricular branch, auricular branch of the vagus nerve, the facial nerve, the auriculotemporal nerve etc.). In some embodiments, the vagus nerve is not stimulated and instead, for example, another nerve is stimulated (e.g., trigeminal nerve, great auricular nerve, the facial nerve, the auriculotemporal nerve, other nerves of the auricular branch, etc.). An auricular (e.g., ear) device can include an ear piece or bud for one or more portions of the ear such as an ear canal or external ear. One to six or more electrodes may be placed on the ear piece or bud, or on a device connected to the ear piece/bud. Right, left or two earpieces are provided in some embodiments. One or more of the vagus, auriculotemporal, trigeminal or cranial nerves may be treated in some embodiments. The device could be unilateral or bilateral, including a single device or multiple devices connected with wires or wirelessly.

In some embodiments, the electrodes themselves are employed as sensing elements (e.g., for measuring nerve activity (e.g., evoked compound action potentials); for detecting electrodermal activity; or cardiac activity; or EEG) and can be placed on or proximate to a subject's wrist or placed on or proximate to a different portion of the subject's body (such as the ear, finger, portion of an arm, etc.).

The device could be unilateral or bilateral, including a single device or multiple devices connected with wires or wirelessly. Transcutaneous neuromodulation is provided in several embodiments, although subcutaneous and percutaneous components may also be used.

Systems with compact, ergonomic form factors are needed to enhance efficacy, compliance, and/or comfort when using non-invasive or wearable neuromodulation devices. In several embodiments, neuromodulation systems and methods are provided that enhance or inhibit nerve impulses and/or neurotransmission, and/or modulate excitability of nerves, neurons, neural circuitry, and/or other neuroanatomy that affects activation of nerves and/or neurons. For example, neuromodulation (e.g., neurostimulation) can include one or more of the following effects on neural tissue: depolarizing the neurons such that the neurons fire action potentials; hyperpolarizing the neurons to inhibit action potentials; depleting neuron ion stores to inhibit firing action potentials; altering with proprioceptive input; influencing muscle contractions; affecting changes in neurotransmitter release or uptake; and/or inhibiting firing.

Stimulation of peripheral nerves can provide therapeutic benefit across a variety of diseases, including but not limited to movement disorders (including but not limited to essential tremor, Parkinson's tremor, orthostatic tremor, and multiple sclerosis), urological disorders, gastrointestinal disorders, cardiac diseases, inflammatory diseases (for example neuroinflammation), mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy is treated in one embodiment. Inflammatory skin conditions and immune dysfunction are also treated in some embodiments. In some embodiments, provided herein are treatments of restless leg syndrome, periodic limb movement disorder, repetitive movements of the limbs and abnormal sensation. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s).

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation, headache, back pain, joint pain and stiffness, muscle pain and tension, etc.

In several embodiments, one or more of bradykinesia, dyskinesia, gait dysfunction, dystonia and/or rigidity are treated. These may be treated in connection with Parkinson's disease or in connection with other disorders. Rehabilitation of movement is treated in some embodiments (for example to restore or improve movement and motion) in subjects who have suffered from an acute or chronic event including, for example, cardiac events (such as atrial fibrillation, hypertension, and stroke), inflammation, neuroinflammation, etc.). Epilepsy is treated in one embodiment.

Other disorders can also be treated through peripheral nerve neurostimulation. For example, stimulation of the sacral and/or tibial nerve has been shown to improve symptoms of overactive bladder and urinary incontinence, and stimulation of the vagus nerve has been shown to improve symptoms of hypertension and cardiac dysrhythmias.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, great auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods describes herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

In some embodiments, wearable systems and methods as disclosed herein can advantageously be used to identify whether a treatment is effective in significantly reducing or preventing a medical condition, including but not limited to tremor severity. Although tremor is treated in several embodiments, the devices described herein are used to treat conditions other than tremor.

Wearable sensors can advantageously monitor, characterize, and aid in the clinical management of hand tremor as well as other medical conditions including those disclosed elsewhere herein. Clinical ratings of medical conditions, e.g., tremor severity can correlate with simultaneous measurements of wrist motion using inertial measurement units (IMUs). For example, tremor features extracted from IMUs at the wrist can provide characteristic information about tremor phenotypes that may be leveraged to improve diagnosis, prognosis, and/or therapeutic outcomes. Kinematic measures can correlate with tremor severity, and machine learning algorithms incorporated in neuromodulation systems and methods as disclosed for example herein can predict tremor severity.

Non-tremor symptoms and conditions are treated herein according to several embodiments. In other non-tremor embodiments, physiological data including heart rate, blood glucose, blood pressure, respiration rate, body temperature, blood volume, sound pressure, photoplethysmography, electroencephalogram, electrocardiogram, blood oxygen saturation, and/or skin conductance as well as patient data from third party devices can be collected and/or aggregated to improve diagnosis, prognosis, and/or therapeutic outcomes for migraine, depression, and/or Lyme disease. For example, physiological data including respiration rate and heart rate along with data related to sleep patterns and activity level can be collected and/or aggregated to improve diagnosis, prognosis, and/or therapeutic outcomes for depression.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc.

The present disclosure will now be described with reference to the accompanying figures, wherein like numerals refer to like elements throughout. The following description is merely illustrative in nature and is in no way intended to limit the disclosure, its application, or uses. It should be understood that steps within a method may be executed in different order without altering the principles of the present disclosure. Furthermore, the devices, systems, and/or methods disclosed herein can include several novel features, no single one of which is solely responsible for its desirable attributes or which is essential to practicing the devices, systems, and/or methods disclosed herein

### A. Overview of Neuromodulation Devices and Methods

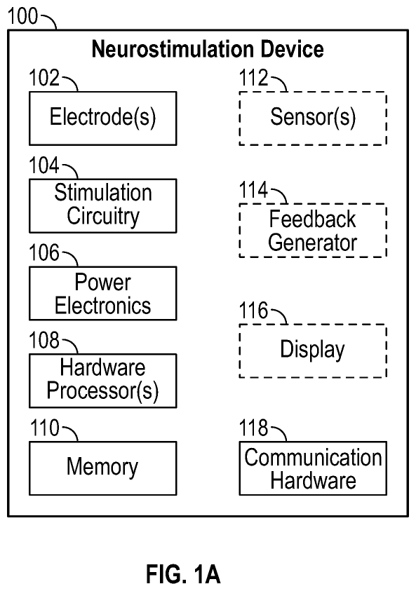

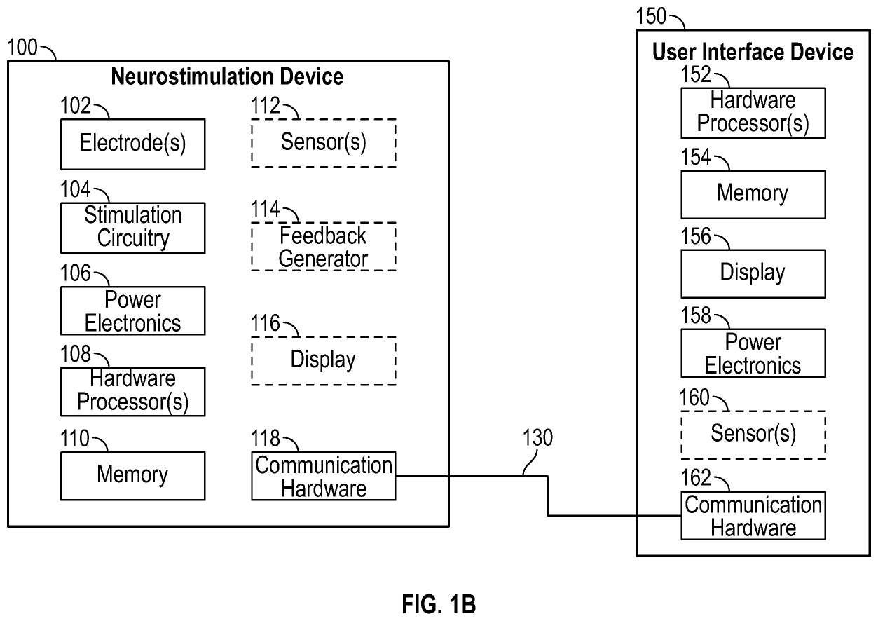

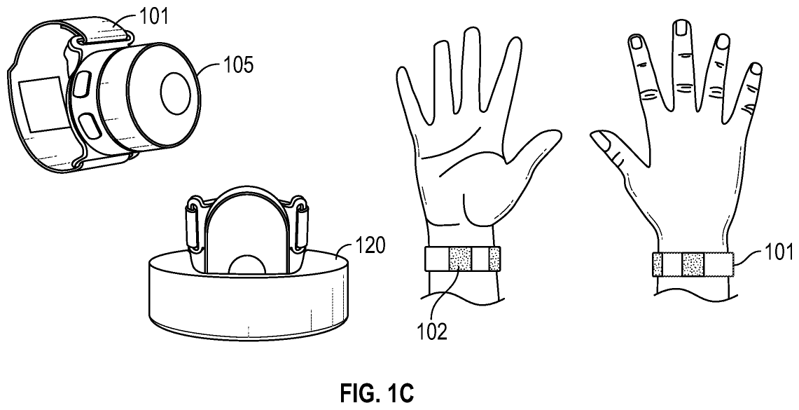

FIG. 1A illustrates a block diagram of an example neuromodulation (e.g., neurostimulation) device 100. The neurostimulation device 100 includes multiple hardware components which are capable of or programmed to provide therapy across the skin of the user. As illustrated in FIG. 1A, some of these hardware components may be optional as indicated by dashed blocks. In some instances, the neurostimulation device 100 may only include the hardware components that are required for stimulation therapy. The hardware components are described in more detail below.

The neurostimulation device 100 can include two or more effectors, e.g. electrodes 102 for providing neurostimulation signals. In some embodiments, the device 100 includes three to six or more electrodes 102 (e.g., 3, 4, 5, 6), and is partially implantable or is entirely transcutaneous. In some embodiments, 2-12 electrodes 102 can be provided (e.g., 2, 3, 4, 5, 6, 7, 8, 9, 10, 11, 12, or more). In some embodiments 3-12 or more electrodes 102 are used (e.g. 3, 6, 9 or 12). In one embodiment, for wrist bands 101, electrodes 102 may not be included on the back of the wrist in one embodiment. Likewise on the ankle, electrodes 102 may be positioned in a manner that enhance patient comfort. Several embodiments provide a wrist worn or ear worn device, or both.

The electrodes 102 could be percutaneous or microneedle electrodes in other embodiments, or only transcutaneous (e.g., not percutaneous, microneedles, or implanted electrodes in some embodiments). In many embodiments, the transcutaneous device 100 is a wearable band or earpiece. The band 101 may partially or fully surround a wrist, finger, arm, leg, ankle or head. Patches may be used, but in many embodiments a patch is not used.

In some embodiments, the electrodes 102 have a generally rectangular shape and includes six electrodes 102. In other embodiments, the electrodes 102 have a round shape or any other shape. Changing the electrode shape can also control the excitation in an area and make the stimulation more comfortable. Square or partially rounded shapes may also be provided. Three to twelve electrodes (e.g., 3, 9, 12 etc.) may be provided in some embodiments. In one embodiment, mechanical (e.g., vibrational) stimulation may be provided before, after or during electrical stimulation for diagnostic and/or therapeutic purposes. Such stimulation may be provided via one or more mechanical/vibratory elements or bands configured to vibrate at a steady or varied frequencies (e.g., of between about 5-50 Hz, 4-60 Hz, 50-100 Hz, 50-300 Hz, 100-450 Hz and overlapping ranges therein). Likewise, the electrical stimulation parameters disclosed herein can be varied or steady within a given time frame (seconds, minutes, hours, etc.). Single or multiple frequencies can be used (e.g., two, three or more electrical stimulations and/or mechanical/vibrational stimulations) at the same, overlapping or different nerves. In one embodiment, varying frequency or other parameters reduces tolerance or habituation and/or increase patient comfort/compliance.

In some embodiments that have six electrodes 102, the six electrodes 102 can be arranged in two sets of three electrodes 102 spaced along the length of the band 101. Of course, the electrodes 102 are not limited to the illustrated shape or number of electrodes 102.

In some instances, the neurostimulation device 100 is configured for transcutaneous use only and does not include any percutaneous or implantable components. In some embodiments, the electrodes can be dry electrodes. In some embodiments, water or gel can be applied to the dry electrode or skin to improve conductance. In some embodiments, the electrodes do not include any hydrogel material, adhesive, or the like. In many embodiments, the transcutaneous device is a wearable band or earpiece. The band may partially or fully surround a wrist, finger, arm, leg, ankle or head. Patches may be used, but in many embodiments a patch is not used.

The neurostimulation device 100 can further include stimulation circuitry 104 for generating signals that are applied through the electrode(s) 102. In some embodiments, the neurostimulation device 100 employs three or more electrodes 102 to apply a stimulation signal to the patient. For example, in some embodiments, at least one electrode is redundant to another electrode (e.g., 2 or more redundant common electrodes and/or 2 or more redundant stimulation electrodes). In this way, even if the electrical contact between one of the two electrodes and the patient's skin is poor increasing resistance, the electrical contact between the redundant electrode and the patient's skin can complete the electrical circuit with a normal or expected level of resistance.

In some embodiments, the 2 or more common electrodes and/or 2 or more stimulation electrodes are circumferentially spaced about the band so that even if the band rotates slightly on the wrist causing an electrode to lose contact with the patient's skin, the redundant electrode will still be in contact with the patient's skin to compete the circuit with a normal or expected level of resistance. In this way, the desired stimulation signal (e.g., frequency, phase, timing, amplitude, and/or offsets) is applied to the patient even when the band rotates on the patient's wrist. The band is less sensitive to electrical contact variations between the electrodes and the patient's skin caused by variations in the angular orientation of the band on the wrist.

The signals can vary in frequency, phase, timing, amplitude, or offsets. The neurostimulation device 100 can also include power electronics 106 for providing power to the hardware components. For example, the power electronics 106 can include a battery.

The neurostimulation device 100 can include one or more hardware processors 108. The hardware processors 108 can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. In an embodiment, all of the processing discussed herein is performed by the hardware processor(s) 108. The memory 110 can store data specific to patient and rules as discussed below.

In the illustrated FIGS. 1A and 1B, the neurostimulation device 100 can include one, two, three, or more sensors 112, which can include any number of combination of inertial measurement units (IMUs) single or multi-axis accelerometers, gyroscopes, inclinometers (to measure and correct for changes in the gravity field resulting from slow changes in the device's orientation), magnetometers, fiber optic electro goniometers, optical tracking or electromagnetic tracking; electromyography (EMG) to detect firing of tremoring muscle; electroneurogram (ENG) signals; cortical recordings by techniques such as electroencephalography (EEG) or direct nerve recordings on an implant in close proximity to the nerve; heart rate or HRV sensors, galvanic skin response sensors (GSR), thermocouples, photoplethysmography sensor (PPG), electrocardiogram sensors (ECG), temperature sensors (e.g., for body/skin temperature or ambient temperature), and/or other physiologic sensors, for example. In some embodiments, the one or more sensors 112 can be employed to measure response to therapy as well as to calibrate therapy. As shown in FIGS. 1A and 1B, the sensor(s) 112 may be optional.

The sensors 112 can include, for example, biomechanical sensors configured to, for example, measure motion, and/or bioelectrical sensors (e.g., EMG, EEG, and/or nerve conduction sensors). The sensors can include, for example, cardiac activity sensors (e.g., ECG, PPG), skin conductance sensors (e.g., galvanic skin response, electrodermal activity), and motion sensors (e.g., accelerometers, gyroscopes). The one or more sensors 112 may include an inertial measurement unit (IMU).

In some embodiments, the IMU can include one or more of a gyroscope, accelerometer, and magnetometer. The IMU can be affixed or integrated with the neuromodulation (e.g., neurostimulation) device 100. In an embodiment, the IMU is an off the shelf component. In addition to its ordinary meaning, the IMU can also include specific components as discussed below. For example, the IMU can include one more sensors capable of collecting motion data. In an embodiment, the IMU includes an accelerometer. In some embodiments, the IMU can include multiple accelerometers to determine motion in multiple axes. Furthermore, the IMU can also include one or more gyroscopes and/or magnetometer in additional embodiments. Since the IMU can be integrated with the neurostimulation device 100, the IMU can generate data from its sensors responsive to motion, movement, or vibration felt by the neurostimulation device 100. Furthermore, when the neurostimulation device 100 with the integrated IMU is worn by a user, the IMU can enable detection of voluntary and/or involuntary motion of the user.

The neurostimulation device 100 can optionally include user interface components, such as a feedback generator 114 and a display 116. The display 116 can provide instructions or information to users relating to calibration or therapy. The display 116 can also provide alerts, such an indication of response to therapy, for example. Alerts may also be provided using the feedback generator 114, which can provide haptic feedback to the user, such as upon initiation or termination of stimulation, for reminder alerts, to alert the user of a troubleshooting condition, to perform a tremor inducing activity to measure tremor motion, among others. Accordingly, the user interface components, such as the feedback generator 114 and the display 116 can provide audio, visual, and haptic feedback to the user.

Furthermore, the neurostimulation device 100 can include communications hardware 118 for wireless or wired communication between the neurostimulation device 100 and an external system, such as the user interface device 150 discussed below. The communications hardware 118 can include an antenna. The communications hardware 118 can also include an Ethernet or data bus interface for wired communications.

While the illustrated figure shows several components of the neurostimulation device 100, some of these components are optional and not required in all embodiments of the neurostimulation device 100. In some embodiments, a system can include a diagnostic device or component that does not include neuromodulation functionality. The diagnostic device could be a companion wearable device connected wirelessly through a connected cloud server, and include, for example, sensors such as cardiac activity, skin conductance, and/or motion sensors as described elsewhere herein.

In some embodiments, the neurostimulation device 100 can also be configured to deliver one, two or more of the following: magnetic, vibrational, mechanical, thermal, ultrasonic, or other forms of stimulation instead of, or in addition to electrical stimulation. Such stimulation can be delivered via one, two, or more effectors in contact with, or proximate the skin surface of the patient. However, in some embodiments, the device is configured to only deliver electrical stimulation, and is not configured to deliver one or more of magnetic, vibrational, mechanical, thermal, ultrasonic, or other forms of stimulation.

Although several neurostimulation devices are described herein, in some embodiments, nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, high frequency stimulation (e.g., greater than about 1 kHz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

### User Interface Devices

FIG. 1B illustrates communications between the neurostimulation device 100 and a user interface device 150 over a communication link 130. The communication link 130 can be wired or wireless. The neuromodulation (e.g., neurostimulation) device 100 is capable of communicating and receiving instructions from the user interface device 150. The user interface device 150 can include a computing device. In some embodiments, the user interface device 150 is a mobile computing device, such as a mobile phone, a smartwatch, a tablet, or a wearable computer. The user interface device 150 can also include server computing systems that are remote from the neurostimulation device. The user interface device 150 can include hardware processor(s) 152, a memory 154, display 156, and power electronics 158. In some embodiments, a user interface device 150 can also include one or more sensors 160, such as sensors described elsewhere herein. Furthermore, in some instances, the user interface device 150 can generate an alert responsive to device issues or a response to therapy. The alert may be received from the neurostimulation device 100 via communication hardware 162.

In additional embodiments, data acquired from the one or more sensors 112 is processed by a combination of the hardware processor(s) 108 and hardware processor(s) 152. In further embodiments, data collected from one or more sensors 112 is transmitted to the user interface device 150 with little or no processing performed by the hardware processors 108. In some embodiments, the user interface device 150 can include a remote server that processes data and transmits signals back to the neurostimulation device 100 (e.g., via the cloud).

The hardware processor 108 and memory 110 can use extracted features from the one or more sensors 112, 160 and determine rules that correspond to neurostimulation therapy. The hardware processor 108 and memory 110 can automatically determine a correlation between specific extracted features and neurostimulation therapy outcomes. Outcomes can include, for example, identifying patients who will respond to the therapy (e.g., during an initial trial fitting or calibration process) based on tremor features of kinematic data (e.g., approximate entropy), predicting stimulation settings for a given patient (based on their tremor features) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient tremor severity at a given point, predicting patient response over time, examining patient medication responsiveness combined with tremor severity over time, predicting response to transcutaneous or percutaneous stimulation, or implantable deep brain stimulation or thalamotomy based off of tremor features and severity over time, and predicting ideal time for a patient to receive transcutaneous or percutaneous stimulation, or deep brain stimulation or thalamotomy based off of tremor features and severity over time, predicting patient reported therapy outcomes or patient reported satisfaction using tremor features assessed kinematic measurements from the device; predicting patient response to undesirable user experience using tremor features assessed from kinematic measurements and patient usage logs from the device where undesirable user experiences can include but are not limited to device malfunctions and adverse events such as skin irritation or burn; predict patient response trends based on tremor severity where trends can be assessed across total number of sessions, within an individual patient, or across a population of patients; predicting or classifying subtypes of tremor to predicting patient response based on kinematic analysis of tremor features; predicting or classifying subtypes of tremor to provide guidance for individually optimized therapy parameters; predicting or classifying subtypes of tremor to optimize the future study design based on subtypes (e.g., selecting specific subtypes of essential tremor for a clinical study with specific design addressing therapy need for the subtype); and predict patient or customer satisfaction (e.g., net promoter score) based on patient response or other kinematic features from measure tremor motion. In some embodiments, essential tremor pathology can include, for example, a primarily cerebellar variant with Bergmann gliosis and Purkinje cell torpedoes, and a Lewy body variant, and a dystonic variant, and a multiple sclerosis variant, and a Parkinson disease variant.

In some embodiments, the neuromodulation, e.g., neurostimulation device 100 can apply transcutaneous stimulation to a patient with tremor that is a candidate for implantable deep brain stimulation or thalamotomy. Tremor features and other sensor measurements of tremor severity will be used to assess response over a prespecified usage period, which could be 1 month or 3 months, or 5, 7, 14, 30, 60, or 90 days or more or less. Response to transcutaneous stimulation as assessed, for example, by algorithms described herein using sensor measurements from the device can advantageously provide input to a predictive model that provides an assessment of the patient's likelihood to respond to implantable deep brain stimulation or other implantable or non-implantable therapies.

In some embodiments, the neuromodulation, e.g., neurostimulation device 100 or a secondary device with sensors can collect motion data, or data from other sensors, when a tremor inducing task is being performed. The patient can be directly instructed to perform the task, for example via the display on the device or audio. In some embodiments, features of tremor inducing tasks are stored on the device and used to automatically activate sensors to measure and store data to memory during relevant tremor tasks. The period of time for measuring and storing data can be, for example, 10, 20, 30, 60, 90, 120 seconds, or 1, 2, 3, 5, 10, 15, 20, 30 minutes, or 1, 2, 3, 4, 5, 6, 7, 8 hours or more or less, or ranges incorporating any two of the foregoing values. Based on a training set of data from a cohort of previous wearers with tremor or another condition, the hardware processor 108 and memory 110 can detect features that are correlated with response to stimulation such that the patient or physician can be presented with a quantitative and/or qualitative likelihood of the patient responding or not responding to treatment. This data can be measured in some cases prior to prescribing the neuromodulation, e.g., neurostimulation or during a trial period. In another embodiment, features can be correlated with the type of tremor measured, such as resting tremor (associated with Parkinson's Disease), postural tremor, action tremor, intention tremor, rhythmic tremor (e.g., a single dominant frequency) or mixed tremor (e.g., multiple frequencies). The type of tremor most likely detected can be presented to the patient or physician as a diagnosis or informative assessment prior to receiving stimulation or to assess appropriateness of prescribing a neuromodulation, e.g., stimulation treatment. In another embodiment, various stimulation modes may be applied based on the tremor type determined; different modes could include changes in stimulation parameters, such as frequency, pulse width, amplitude, burst frequency, duration of stimulation, or time of day stimulation is applied. In one embodiment for a smartphone, tablet, or other device, the task to induce tremor can be included in an app that asks the patient to take a self-photograph, which has the patient perform a task that has both posture and intention actions.

In some embodiments, the neuromodulation, e.g., neurostimulation device 100 or a secondary device with sensors can collect motion data, or data from other sensors, can measure data over a longer period of time, for example 1, 2, 3, 4, 5, 10, 20, 30 weeks, 1, 2, 3, 6, 9, 12 months, or 1, 2, 3, 5, 10 years or more or less, or ranges incorporating any two of the foregoing values, to determine features, or biomarkers, associated with the onset of tremor diseases, such as essential tremor, Parkinson's disease, dystonia, multiple sclerosis, etc. Biomarkers could include specific changes in one or more features of the data over time, or one or more features crossing a predetermined threshold. In some embodiments, features of tremor inducing tasks have been stored on the device and used to automatically activate sensors when those tremor inducing tasks are being performed, to measure and store data to memory during relevant times.

In some embodiments, the hardware processor 108 and memory 110 rely on instructions to determine rules between features and outcomes. The hardware processor 108 and memory 110 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the hardware processor 108 and memory 110 can use the rules to automatically determine outcomes. The hardware processor 108 and memory 110 can also use the rules to control or change settings of the neurostimulation device, including but not limited to stimulation parameters (e.g., stimulation amplitude, frequency, patterned (e.g., burst stimulation), intervals, time of day, individual session or cumulative on time, and the like).

Accordingly, the rules can improve operation of the neuromodulation, e.g., neurostimulation device 100, and advantageously and accurately identify potential candidates for therapy and well as various disease state and therapy parameters over time. The generated rules can be saved in the memory 110 and/or memory 154. For example, the rules can be generated and stored prior to operation of the neurostimulation device 100. Accordingly, in some embodiments, the hardware processor 108 and memory 110 can apply the saved rules on new data collected by the sensors 112, 160 to determine outcomes or control the neuromodulation, e.g., neurostimulation device 100.

In some embodiments, the neuromodulation device 100 can include the ability to track a user's motion data for the purpose of gauging one, two, or more tremor frequencies of a patient. The patient could have a single tremor frequency, or in some cases multiple discrete tremor frequencies that manifest when performing different tasks. Once the tremor frequencies are observed, they can be used as one of many seminal input parameters to a customized neuromodulation therapy.

The therapy can be delivered, e.g., transcutaneously, via one, two, or more nerves (e.g., the median and radial nerves, and/or other nerves disclosed elsewhere herein) in order to reduce or improve a condition of the patient, including but not limited to their tremor burden. In some embodiments, the therapy modulates afferent nerves, but not efferent nerves. In some embodiments, the therapy preferentially modulates afferent nerves. In some embodiments, the therapy does not involve functional electrical stimulation.

Although transcutaneous delivery is used in many embodiments, in some embodiments at least a portion of the devices may be implanted subcutaneously or percutaneously. In one embodiment, a first electrode stimulates the median nerve, a second electrode stimulates the radial nerve, and a third electrode stimulates the ulnar nerve. In one embodiment, two or more electrodes stimulate the same nerve (e.g., with different frequencies or other parameters). In one embodiment, one two or all of the median nerve, radial nerve, and ulnar nerve are stimulated. In some embodiments, the stimulation electrodes themselves are employed as sensing elements (e.g., for measuring nerve activity (e.g., evoked compound action potentials); for detecting electrodermal activity; or cardiac activity; or EEG) and can be placed on or proximate to a subject's wrist or placed on or proximate to a different portion of the subject's body (such as the ear, finger, portion of an arm, etc.). Specific examples for controlling the neurostimulation device 100 are described in more detail below.

FIG. 1C schematically illustrates a neurostimulation device 100 and base station 120. The neurostimulation device 100 can include a stimulator 105 and detachable band 101 including two or more working electrodes 102 (positioned over the median and radial nerves) and a counter-electrode 102 positioned on the dorsal side of the wrist. The electrodes could be, for example, dry electrodes or hydrogel electrodes. The base station 120 can be configured to stream movement sensor and usage data on a periodic basis, e.g., daily and charge the neurostimulation device 100. The neurostimulation device 100 stimulation bursting frequency can be calibrated to a lateral postural hold task “wing-beating” or forward postural hold task for a predetermined time, e.g., 10, 20, 30 seconds for each subject. Other non-limiting examples of neurostimulation device 100 parameters can be as disclosed elsewhere herein.

In some embodiments 3-12 or more electrodes are used (e.g. 3, 6, 9 or 12). In one embodiment, none of the electrodes are in contact with areas that cause discomfort. In many embodiments, the transcutaneous device is a wearable band or earpiece. The band may partially or fully surround a wrist, finger, arm, leg, ankle or head. Patches may be used, but in many embodiments a patch is not used.

In some embodiments, stimulation may alternate between each nerve such that the nerves are not stimulated simultaneously. In some embodiments, all nerves are stimulated simultaneously. In some embodiments, stimulation is delivered to the various nerves in one of many bursting patterns. For example, the bursting patterns can include variations in stimulation parameters including, for example, on/off, time duration, intensity, pulse rate, pulse width, waveform shape, and the ramp of pulse on and off. In one embodiment the pulse rate may be from about 1 to about 5000 Hz, about 1 Hz to about 500 Hz, about 5 Hz to about 50 Hz, about 50 Hz to about 300 Hz, or about 150 Hz, and overlapping ranges therein. In some embodiments, the pulse rate may be from 1 kHz to 20 KHz. In some embodiments, a pulse width may range from, in some cases, 50 to 500 μs (micro-seconds), such as approximately 50-150, 150-300, 300-500, such as 100, 200, 300, 400 μs, and overlapping ranges therein. Although frequencies below 5 kHz are used in several embodiments, some embodiments use higher frequency stimulation (e.g., of nerves at or near the wrist or ear) of 5-75 kHz (e.g., 10-40 KHz, 15-60 KHz, etc.) and a pulse width of 1-20, 10-50, 10-40 μs. The intensity of the electrical stimulation may vary from 0 mA to 500 mA, and a current may be approximately 1-11, 1-20, 5-50, 10-100 mA, and overlapping ranges therein. The electrical stimulation can be adjusted in different patients and with different methods of electrical stimulation. The increment of intensity adjustment may be, for example, 0.1 mA to 1.0 mA, such as 0.1-0.5, 0.5-0.75, 5-1 mA, and overlapping ranges therein. In some embodiments, the stimulation may last for approximately 10 minutes to 1 hour, such as approximately 10, 20, 30, 40, 50, or 60 minutes, or ranges including any two of the foregoing values. In some embodiments, stimulation may be provided for 30, 40, 50, 60, 80, 90, 120, 150 minutes 1-4 times a day. In some embodiments, stimulation occurs for 2-15 minutes (e.g., 3, 5, 7, 10 minutes) every hour (or on another interval) for a total of 40-240 minutes (e.g., 60, 80, 90, 120, 150 minutes) in a 12 or 24 hour period. Differing dosing schedules and/or differing stimulation parameters may reduce tolerance or habituation and/or may increase patient comfort/compliance. In one embodiment, beneficial effects of stimulation are provided during off periods; for example, a patient's tremor or other symptom/indication is reduced because the prior stimulation results in a prolonged effect on the nerve(s). Thus, a patient may be able to reduce the length, duration etc. of therapy over time. Burst patterns include but are not limited to theta burst stimulation.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, high frequency stimulation (e.g., greater than about 1 kHz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

In some embodiments, a plurality of electrical stimuli can be delivered offset in time from each other by a predetermined fraction of multiple of a period of a measured rhythmic biological signal such as hand tremor, such as about ¼, ½, or ¾ of the period of the measured signal for example. Further possible stimulation parameters are described, for example, in U.S. Pat. No. 9,452,287 to Rosenbluth et al., U.S. Pat. No. 9,802,041 to Wong et al., PCT Pub. No. WO 2016/201366 to Wong et al., PCT Pub. No. WO 2017/132067 to Wong et al., PCT Pub. No. WO 2017/023864 to Hamner et al., PCT Pub. No. WO 2017/053847 to Hamner et al., PCT Pub. No. WO 2018/009680 to Wong et al., PCT Pub. No. WO 2018/039458 to Rosenbluth et al., PCT Pub. No. WO 2018/187241 to Hamner et al., PCT Pub. No. WO 2019/213433 to Liberatore et al., PCT Pub. No. WO 2020/006048 to Rosenbluth et al., PCT Pub. No. WO 2020/069219 to Ross et al., PCT Pub. No. WO 2020/086726 to Hamner et al., PCT Pub. No. WO 2020/185601 to Hamner et al., PCT Pub. No. WO 2021/0252278 to Hamner et al., and PCT Pub. No. WO 2021/236815 to Kent et al., each of the foregoing of which are hereby incorporated by reference in their entireties.

### B. Example Electrode Configurations

Some previously described neuromodulation (e.g., neurostimulation) device describe using multiple electrodes, such as at least three electrodes, in order to stimulate multiple peripheral nerves, such as various combinations of the median, radial, and ulnar nerves in the arm. For example, each nerve can be stimulated with a dedicated electrode pair, which would require twice the number of electrodes compared to the number of nerves to be stimulated. For example, to stimulate both radial and median nerves would require 4 electrodes. Circumferential electrodes with a dedicated return electrode and individual electrodes placed over each nerve could also be used. For example, three circumferentially spaced electrodes can also be used to stimulate those two nerves, thereby reducing the number of electrodes by one, to three electrodes.

It is also possible to further reduce the number of electrodes required to stimulate multiple nerves in order to reduce the size and cost of the stimulation surface or disposable. In some embodiments, a stimulator with N electrodes (e.g., where N is an integer greater than 1, e.g., 2 or at least 2 electrodes) can be used to stimulate exactly N nerves (e.g., where N is an integer greater than 1, e.g., 2 or at least 2 electrodes) either simultaneously or in an alternating pattern to treat various disorders such as, for example, tremor, overactive bladder, hypertension, arrhythmias, and other conditions. For example, in some embodiments, two nerves, such as the median and radial nerves, can be stimulated using exactly two electrodes, rather than the three or more electrodes as described above and as described in International Application Number PCT/US2015/033809 (International Publication Number WO2015/187712) and U.S. patent application Ser. No. 14/805,385 (U.S. Application Publication No. 2015/0321000), which are each incorporated by reference in their entireties. In other embodiments, exactly 3 nerves can be stimulated by exactly 3 electrodes, exactly 4 nerves can be stimulated by exactly 4 electrodes, etc. Further possible stimulation parameters are described, for example, PCT Pub. No. WO 2018/009680 to Wong et al., which is hereby incorporated by reference in its entirety.

The placement and configuration of the electrodes used to stimulate multiple nerves may affect a user's comfortability when the electrodes are placed on the user's skin. For example, when the circumferential electrodes are placed around a user's wrist, one or more stimulating electrodes may be placed at the ventral side of the user's wrist because some of the target nerves to be stimulated are close to the ventral side of the wrist, thus, their corresponding counter electrodes may be placed on the dorsal side of the user's wrist. In some situations, although a band or strip may tightly place the electrodes on the skin of the patient's wrist, the electrodes on the dorsal side of the patient's wrist may be raised partially above the skin, which results in discomfort during the wrist motion. Moreover, the configuration and placement of the electrodes may also affect the spread of the electrical current during nerve stimulation.

Bipolar stimulating electrodes that are used for non-invasive peripheral nerve stimulation may be placed differently in different embodiments. In some embodiments, stimulating and counter electrodes may be spaced circumferentially. In some embodiments, circumferential electrode configuration means that the stimulating and counter electrodes are placed on the opposite sides of a limb with the target nerve between the stimulating electrode and counter electrode. In some embodiments, the configuration of electrodes may be concentric. In some embodiments, concentric electrode configuration means that the counter electrode surrounds the stimulating electrode or vice versa. In some embodiments, the configuration of electrodes may be in-line. In some embodiments, in-line electrode configuration means that one or more stimulating electrodes and one or more counter electrodes align along the target nerve when placed on a user's skin.

### 1. Example Circumferentially Spaced Electrodes

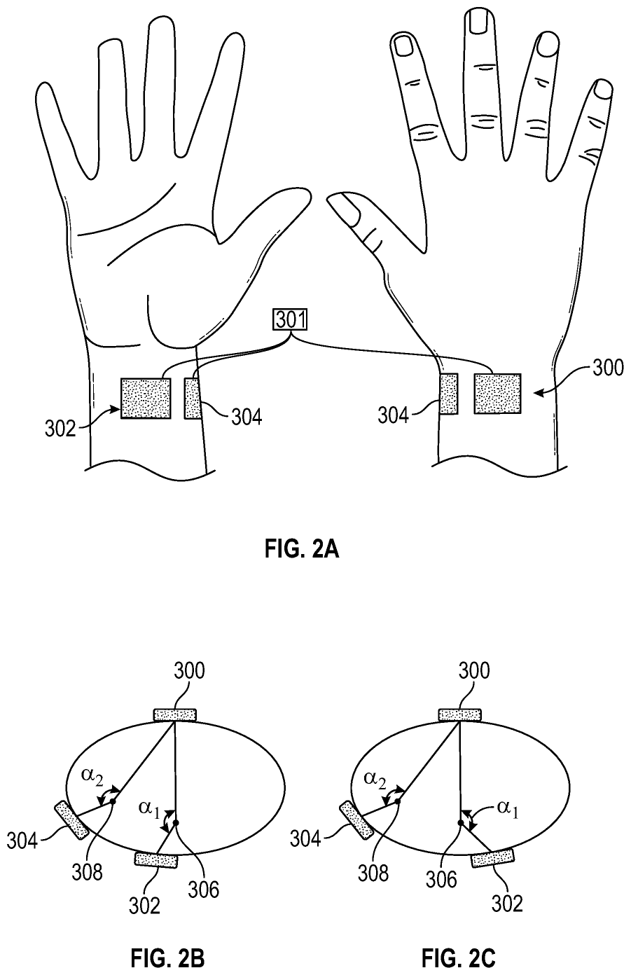

One aspect of the neuromodulation (e.g., neurostimulation) device, as schematically illustrated in FIGS. 2A-2C, is the use of only three electrodes to electrically stimulate two nerves (e.g., median and radial), with an electrode 302, 304 placed on the skin over or proximate to each one of the two nerves 306, 308 and a third charge balance electrode 300 placed on an opposite side of the body part (e.g., wrist) as the two nerves 306, 308. FIG. 2A shows the dorsal side (left) and ventral side (right) of a user's wrist and illustrates an example of the placement of the three electrodes 300, 302, 304 on the user's wrist for targeting two nerves. The three electrodes 300, 302, 304 may all be operatively connected to a single controller 301, as schematically illustrated in FIG. 2A, for regulating the target stimulation of the nerves. In some embodiments, the third electrode 300 (e.g., a charge balance electrode) can be placed approximately on the longitudinal midline of the dorsal side of the arm or wrist. In some embodiments, the first electrode 302 can be placed approximately on the longitudinal midline of the ventral side of the arm or wrist to target the median nerve. In some embodiments, the second electrode 304 can be placed in between the charge balance electrode 300 and the ventrally placed electrode 302 to target the radial nerve. In some embodiments, yet another electrode (not shown) can be placed to target the ulnar nerve or an electrode targeting the ulnar nerve can replace either the first electrode 302 targeting the median nerve 306 or the second electrode 304 targeting the radial nerve 308.

FIGS. 2B and 2C illustrate the positions of the charge balance electrode 300, the ventrally placed electrode 302, and the radial electrode 304 in relation to the median nerve 306 and the radial nerve 308 in a distal-looking transverse cross-sectional plane of the patient's wrist or arm. The electrodes 300, 302, 304 are positioned such that in a projection into the transverse cross-sectional plane of the arm or wrist there is a 90 degree to 180 degree angle, α1, between a line connecting the median nerve 306 and the center of the charge balance electrode 300 and a line connecting the median nerve 306 and the center of the ventrally placed electrode 302, and there is a 90 degree to 180 degree angle, α2, between a line connecting the radial nerve 308 and the charge balance electrode 300 and a line connecting the radial nerve 308 and the radial electrode 304. The angles α1 and α2 may each be measured in either a counter-clockwise direction (as α1 is shown in FIG. 2B) or in a clockwise direction (as α1 is shown in FIG. 2C). More generally, the electrodes 300, 302, 304 can be spaced apart by a predetermined distance such that when the electrodes 300, 302, 304 are positioned circumferentially around a patient's wrist, one of the angles formed between each electrode pair and its target nerve is between about 90 degrees and 180 degrees. Such an orientation results in each electrode of the electrode pair being placed generally on opposite sides of the target nerve. In other words, the target nerve is positioned approximately between the electrode pair.

Although the embodiments have been described with reference to three electrodes for the stimulation of two nerves, it is understood that alternative embodiments can utilize two electrodes to stimulate a single nerve, where the two electrodes can have a fixed spacing to allow the electrodes to stimulate the nerve from opposing sides of the nerve. Similarly, other embodiments can utilize more than three electrodes. For instance, an additional electrode can be added to target the ulnar nerve. In some embodiments, five or more electrodes may be used to target four or more nerves. In addition, different combination of electrodes can be used to target one or more nerves from the group of the median, radial, and ulnar nerves.

Mapping the nerves of a number of individuals with different wrist sizes was performed by selectively stimulating circumferential locations on the wrist and verifying where the user feels paresthesia in order to identify the median, radial, and ulnar nerve. The mapping showed variability in nerve location relative to wrist size, as well as high individual variability in physiology. Individual nerves can be target with electrodes positioned at the correct location, such as the positions shown in FIG. 2A, or by using an array of multiple electrodes allowing selection of electrodes which target those individual nerves, as discussed elsewhere herein.

More detailed description of circumferential electrode configuration and nerve stimulation may be found for example, in International Application No. PCT/US2017/040920 (International Publication Number WO2018/009680), U.S. Pat. No. 10,814,130, International Application Number PCT/US2015/033809 (International Publication Number WO2015/187712) and U.S. patent application Ser. No. 14/805,385 (U.S. Application Publication No. 2015/0321000), which are each incorporated by reference in their entireties.

### 2. Example Concentric Electrode Configuration

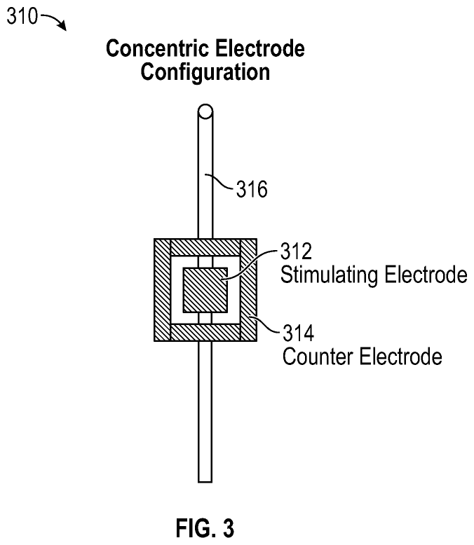

In one aspect, the neuromodulation (e.g., neurostimulation) device includes one or more concentric electrode systems to electrically stimulate each target nerve (e.g., median, ulnar and/or radial nerves). FIG. 3 illustrates an example embodiment of a concentric electrode system 310. The concentric electrode system 310 may be a two-electrode system. In some embodiments, the concentric electrode system 310 may include a stimulating electrode 312 and a counter electrode 314. The stimulating and the counter electrodes may share the same center. The concentric electrode system 310 may be placed on a patient's skin over or proximate to a target nerve 316. The target nerve 316 may be any of the nerve that the device designed to stimulate, such as median, radial, or ulnar nerves in the wrist or fingers for treating diseases, including but not limited to movement disorders (including but not limited to essential tremor, Parkinson's tremor, orthostatic tremor, and multiple sclerosis), urological disorders, gastrointestinal disorders, cardiac diseases, and inflammatory diseases, mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), among others. Tourette's and other involuntary or undesired tic or movement is treated in some embodiments.

In some embodiments, the edge-to-edge gap between the stimulating electrode 312 and the counter electrode 314 may be about 0.5 mm to about 5 mm. In some embodiments, the edge-to-edge gap between the stimulating electrode 312 and the counter electrode 314 may be about 0.5 mm, about 1 mm, about 1.5 mm, about 2 mm, about 3 mm, about 4 mm, about 5 mm, or any other distance. In some embodiments, the stimulating electrode 312 in the concentric electrode system 310 may be a square, rectangular, ellipse, ring, or any other shape. In some embodiments, the length of each side of a square stimulating electrode 312 may be about 10 mm to about 30 mm. In some embodiments, the length of each side of a square stimulating electrode 312 may be about 10 mm, about 15 mm, about 20 mm, about 25 mm, about 30 mm, or any other length. In some embodiments, the strip width of the counter electrode 304 may be about 1 mm to about 10 mm. In some embodiments, the strip width of the counter electrode 314 may be about 1 mm, about 2 mm, about 3 mm, about 4 mm, about 5 mm, about 6 mm, about 7 mm, about 8 mm, about 9 mm, about 10 mm, or any other width. In some embodiments, the stimulating electrode may surrounds the counter electrode, for example, the electrode 312 may be a counter electrode and the electrode 314 may be a stimulating electrode.

In some embodiments, one concentric electrode system, such as system 310, may be used to stimulate one target nerve. In some embodiments there may be one, two three, or more concentric electrode systems in a neuromodulation (e.g., neurostimulation) device. The number of concentric electrode system depends, at least partially, on the number of target nerves. In some embodiments, a neuromodulation (e.g., neurostimulation) device may include one, or two, or three, or more concentric electrode system 310 to stimulate one, or two, or three, or more nerves.

One advantage of the concentric electrode system is that the neural activation thresholds may be reduced comparing to a conventional electrode placement and configuration. The concentric electrode system may be placed on a user's skin close to the target nerve and the spread of electrical current during nerve stimulation may be constrained around the concentric electrode system. Thus, the neural activation thresholds may be reduced. Moreover, the comfortability of wearing a device with such concentric electrode systems may be improved as there may be no need to place counter electrode on the dorsal side of a user's wrist. For example, when the circumferential spaced electrodes are placed around a user's wrist, one or more counter electrodes may be placed on the dorsal side of the user's wrist, e.g., the electrode 300 in FIG. 2A. Although the electrodes a band or strip may tightly attach the electrodes to the skin of the patient's wrist, in some situations, the electrodes on the dorsal side of the user's wrist may be raised partially above the skin and result in discomfort during the wrist motion. Without the necessity of placing a counter electrode on the dorsal side of the user's wrist, the concentric electrode system may improve the comfortability of wearing such a device. In addition, the concentric electrode system could allow integration with other wearable devices (e.g., a smart watch display) by not requiring an electrode under the other wearable device facing the dorsal side of the wrist.

### 3. Example In-Line Electrode Configuration

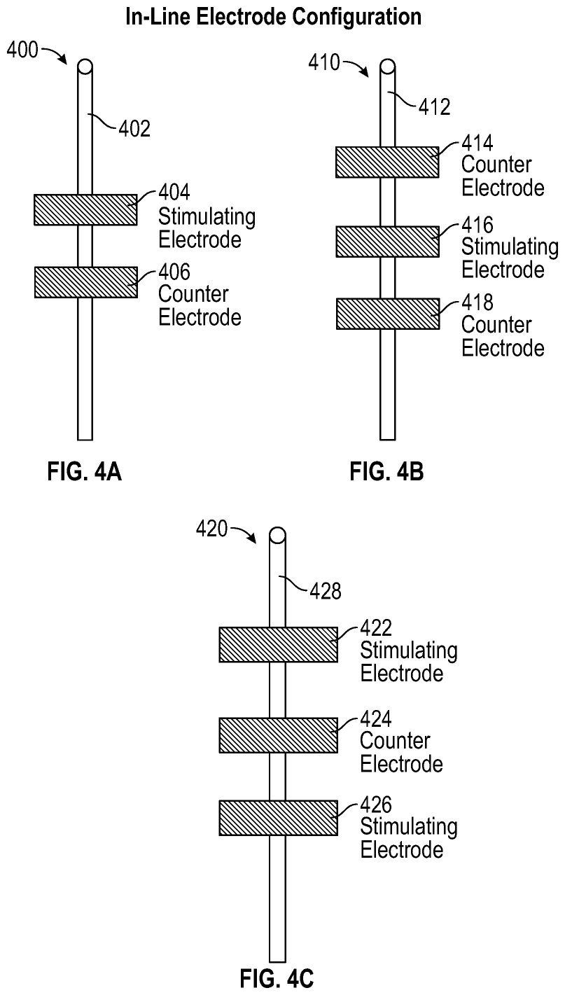

One aspect of the neuromodulation (e.g., neurostimulation) device is the incorporation of an in-line electrode system to electrically stimulate a nerve in close proximity of the in-line electrode system. FIGS. 4A-C illustrate various example embodiments of an in-line electrode system to deliver electrical current to stimulate a target nerve.

FIG. 4A schematically illustrates an example of a two-electrode in-line electrode system 400 targeting one nerve. The two-electrode in-line electrode system 400 may be placed on a patient's skin over or proximate to a target nerve 402. The target nerve 402 may be a nerve that the device is designed to stimulate, such as a ulnar, median and/or radial nerve. The in-line electrode system 400 may include a stimulating electrode 404 and a counter electrode 406. In some embodiments, the stimulating electrode 404 and counter electrode 406 may be longitudinally placed along the same nerve 402 on the same side of the wrist relative to the target nerve. The edge-to-edge gap between the stimulating electrode 404 and the counter electrode 406 may be about 0.5 mm to about 5 mm. In some embodiments, the edge-to-edge gap between the stimulating electrode 404 and the counter electrode 406 may be about 0.5 mm, about 1 mm, about 1.5 mm, about 2 mm, about 3 mm, about 4 mm, about 5 mm, or any other distance.

In some embodiments, the stimulating electrode 404 may have a length of about 10 mm to about 30 mm. In some embodiments, the length of the stimulating electrode 404 and/or counter electrode 406 may be about 10 mm, about 12 mm, about 15 mm, about 20 mm, about 22 mm, about 30 mm, or any other length. In some embodiments, the strip width of the counter electrode 406 and/or stimulating electrode 404 may be about 1 mm to about 10 mm. In some embodiments, the strip width of the counter electrode 406 and/or stimulating electrode may be about 1 mm, about 2 mm, about 3 mm, about 4 mm, about 5 mm, about 6 mm, about 7 mm, about 8 mm, about 9 mm, about 10 mm, or any other width.

In some embodiments, the size and placement of the stimulating electrode and/or counter electrode may depend, at least partially, on the width of the band to improve the comfortability for a user when wearing the device. In some embodiments, the size of the stimulating electrode 404 may be the same as the size of the counter electrode 406. In some embodiments, the size of the stimulating electrode 404 may be different from the size of the counter electrode 406. In some examples, the size and placement of the stimulating electrode and/or counter electrode may depend, at least partially, on the effect of the stimulation to activate the nerves. In some examples, the size and placement of the stimulating electrode and/or counter electrode may be configured to achieve a balance between comfortability and effect of stimulation.

FIG. 4B illustrates another example embodiment of an in-line electrode system 410. The in-line electrode system 410 may be a three-electrode system. In some embodiments, the in-line electrode system 410 may include a counter electrode 414, a stimulating electrode 416, and a counter electrode 418. The in-line electrode system 410 may be placed on a patient's skin over or proximate to a target nerve 412. The target nerve may the nerve that the device designed to stimulate, such as a median, ulnar and/or radial nerve. In some embodiments, the stimulating electrode 416 may be placed between the counter electrode 414 and counter electrode 418. In some embodiments, the counter electrode 414, the stimulating electrode 416, and the counter electrode 418 may be longitudinally placed along the same nerve 412 on the same side of the arm relative to the target nerve.

In some embodiments, the edge-to-edge gap between the stimulating electrode 416 and the counter electrode 414 may be about 0.5 mm to about 5.0 mm. In some embodiments, the edge-to-edge gap between the stimulating electrode 416 and the counter electrode 414 may be about 0.5 mm, about 1 mm, about 1.5 mm, about 2 mm, about 3 mm, about 4 mm, about 5 mm, or any other distance. In some embodiments, the edge-to-edge gap between the stimulating electrode 416 and the counter electrode 418 may be about 0.5 mm to about 5 mm. In some embodiments, the edge-to-edge gap between the stimulating electrode 416 and the counter electrode 418 may be about 0.5 mm, about 1 mm, about 1.5 mm, about 2 mm, about 3 mm, about 4 mm, about 5 mm, or any other distance. In some embodiments, the gap between the counter electrode 414 and stimulating electrode 416 and the gap between the counter electrode 418 and the stimulating electrode 416 may be the same. In some embodiments, the gap between the counter electrode 414 and stimulating electrode 416 and the gap between the counter electrode 418 and the stimulating electrode 416 may be the different.

In some embodiments, the stimulating electrode 416, the counter electrode 414, and/or the counter electrode 418 may have a length of about 10 mm to about 30 mm. In some embodiments, the length of the stimulating electrode 416, the counter electrode 414, and/or the counter electrode 418 may be about 10 mm, about 12 mm, about 15 mm, about 20 mm, about 22 mm, about 30 mm, or any other length. In some embodiments, the strip width of the stimulating electrode 416, the counter electrode 414, and/or the counter electrode 418 may be about 1 mm to about 10 mm. In some embodiments, the strip width of the stimulating electrode 416, the counter electrode 414, and/or the counter electrode 418 may be about 1 mm, about 2 mm, about 3 mm, about 4 mm, about 5 mm, about 6 mm, about 7 mm, about 8 mm, about 9 mm, about 10 mm, or any other width. In some embodiments, the counter electrode 414 is the same as counter electrode 418. In some embodiments, the counter electrode 414 and counter electrode 418 may have different sizes. In some embodiments, the size of the stimulating electrode 416 is the same as size of the counter electrode 414 and/or counter electrode 418. In some embodiments, the size of the stimulating electrode 416 may be different from the size of the counter electrode 414 and/or counter electrode 418.

In some embodiments, the size and placement of the stimulating electrode and/or counter electrodes may depend, at least partially, on the width of the band to improve the comfortability for a user when wearing the device. In some examples, the size and placement of the stimulating electrode and/or counter electrodes may depend, at least partially, on the effect of the stimulation to activate the nerves. In some examples, the size and placement of the stimulating electrode and/or counter electrodes may be configured to achieve a balance between comfortability and effect of stimulation.

FIG. 4C schematically illustrates another example embodiment of an in-line electrode system 420. The in-line electrode system 420 may be a three-electrode system. In some embodiments, the in-line electrode system 420 may include a stimulating electrode 422, a counter electrode 424, and a stimulating electrode 426. The in-line electrode system 420 may be placed on a patient's skin over or proximate to a target nerve 428. The target nerve 428 may be any of the nerve that the device wants to stimulate, such as an ulnar, median and/or radial nerve. In some embodiments, the counter electrode 424 may be placed between stimulating electrodes 422 and 426. In some embodiments, the counter electrode 424, the stimulating electrodes 422 and 426 may be longitudinally placed along the same nerve 428 on the same side of the arm relative to the target nerve.

In some embodiments, the edge-to-edge gap between the stimulating electrode 422 and the counter electrode 424 may be about 0.5 mm to about 5 mm. In some embodiments, the edge-to-edge gap between the stimulating electrode 422 and the counter electrode 424 may be about 0.5 mm, about 1 mm, about 1.5 mm, about 2 mm, about 3 mm, about 4 mm, about 5 mm, or any other distance. In some embodiments, the edge-to-edge gap between the stimulating electrode 426 and the counter electrode 424 may be about 0.5 mm to about 5 mm. In some embodiments, the edge-to-edge gap between the stimulating electrode 426 and the counter electrode 424 may be about 0.5 mm, about 1 mm, about 1.5 mm, about 2 mm, about 3 mm, about 4 mm, about 5 mm, or any other distance.

In some embodiments, the counter electrode 424, the stimulating electrodes 422 and/or the stimulating electrode 426 may have a length of about 10 mm to about 30 mm. In some embodiments, the length of the counter electrode 424, the stimulating electrodes 422 and/or the stimulating electrode 426 may be about 10 mm, about 12 mm, about 15 mm, about 20 mm, about 22 mm, about 30 mm, or any other length. In some embodiments, the strip width of the counter electrode 424, the stimulating electrode 422, and/or stimulating electrode 426 may be about 1 mm to about 10 mm. In some embodiments, the strip width of the counter electrode 424, stimulating electrode 422, and/or stimulating electrode 426 may be about 1 mm, about 2 mm, about 3 mm, about 4 mm, about 5 mm, about 6 mm, about 7 mm, about 8 mm, about 9 mm, about 10 mm, or any other width.

In some embodiments, the size and placement of the stimulating electrodes and/or counter electrode may depend, at least partially, on the width of the band to improve the comfortability for a user when wearing the device. In some examples, the size and placement of the stimulating electrode and/or counter electrode may depend, at least partially, on the effect of the stimulation to activate the nerves. In some examples, the size and placement of the stimulating electrode and/or counter electrodes may be configured to achieve a balance between comfortability and effect of stimulation.

In some embodiments, one in-line electrode system, such as in-line electrode system 400, 410, and/or 420, may be used to stimulate one target nerve. In some examples, there may be one, two three, or more in-line electrode systems in a neuromodulation (e.g., neurostimulation) device, which depends, at least partially, on the number of target nerves to be stimulated. In some embodiments, a neuromodulation (e.g., neurostimulation) device may include one, or two, or three, or more in-line electrode systems to stimulate one, or two, or three, or more nerves. In some embodiments, the spacing of the in-line electrode systems depends, at least in part, on the spacing between the nerves to be stimulated. In some embodiments, the spacing of the in-line electrode systems depends, at least in part, on the desired levels of stimulation for the nerves.

Similar to a concentric electrode system, one advantage of the in-line electrode system may be that the neural activation thresholds may be reduced comparing to a conventional electrode placement and configuration. The in-line electrode system may be placed on a user's skin close to the target nerve and the spread of electrical current during nerve stimulation may be constrained around the in-line electrode system. Thus, the neural activation thresholds may be reduced. Moreover, the comfortability of wearing a device with such in-line electrode systems may be improved as there may be no need to place counter electrode on the dorsal side of a user's wrist. In contrast, when the circumferential spaced electrodes are placed around a user's wrist, one or more counter electrodes may be placed on the dorsal side of the user's wrist, e.g., the electrode 300 in FIG. 2A. Although the electrodes may be tightly attached to the skin of the patient's wrist by a band or strip, in some situations, the electrodes on the dorsal side of the user's wrist may be raised partially above the skin and result in discomfort during the wrist motion. Without the necessity of placing a counter electrode on the dorsal side of the user's wrist, the in-line electrode system may improve the comfortability of wearing such a device. In addition, the in-line electrode system may allow the integration with other wearable devices (e.g., a smart watch display) by not requiring an electrode under the device face on the dorsal side of the wrist.

Moreover, comparing to the in-line electrode configurations, the concentric electrode configuration may require more thoughtful placement of the electrode system relative to the target nerves to ensure nerve activation. The in-line electrode configurations may allow more flexibility in electrode placement. In some embodiments, the amplitude required to feel paresthesia may be reduced with the in-line configuration, compared to the concentric configuration, suggesting that the in-line electrode configuration targets the nerve more efficiently.

### C. Example Stimulation Waveform and Stimulation Timing

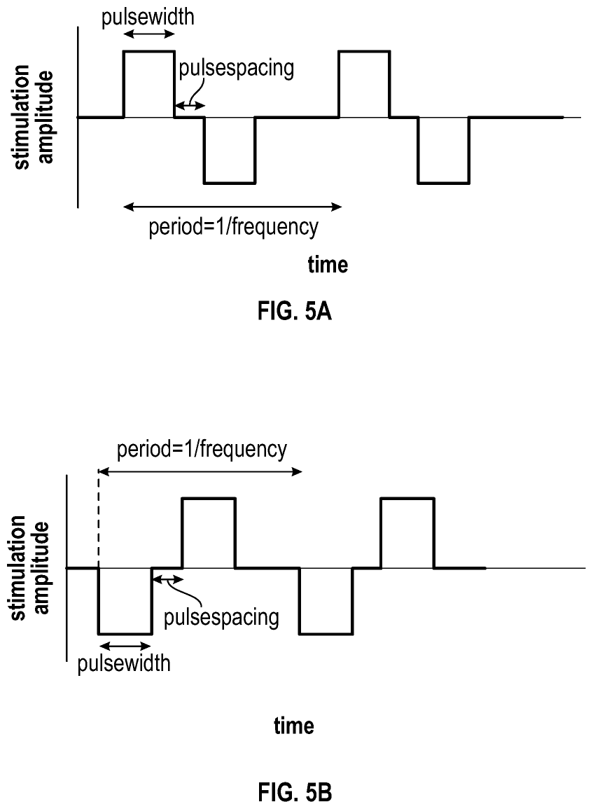

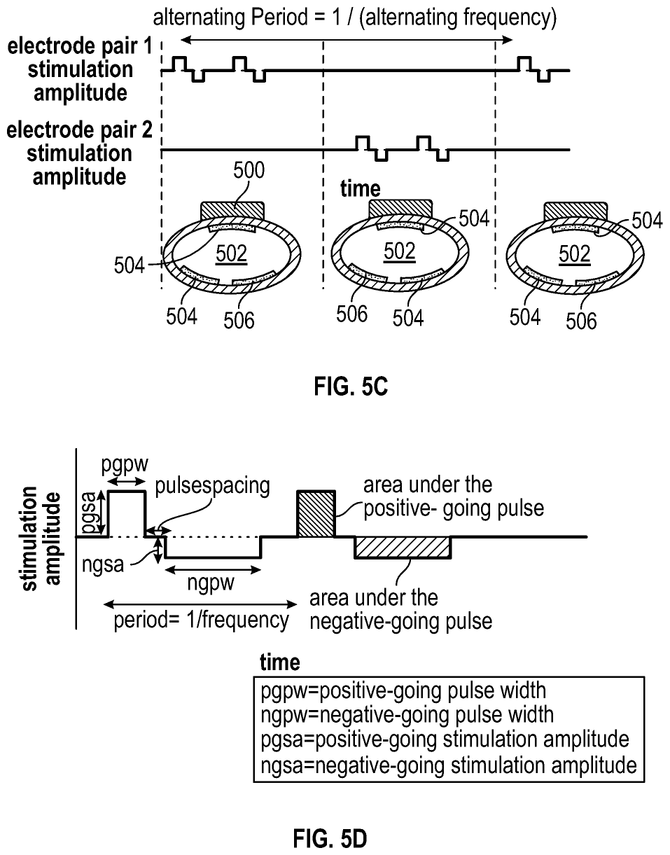

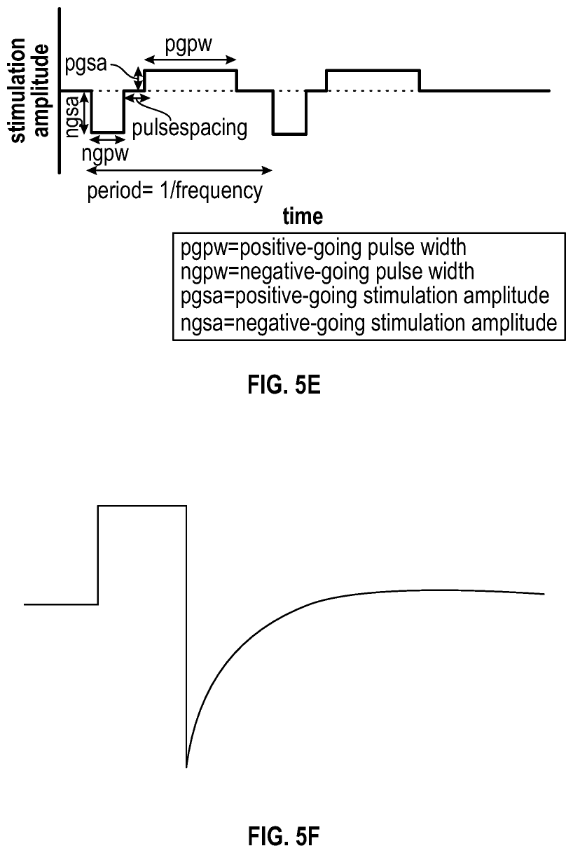

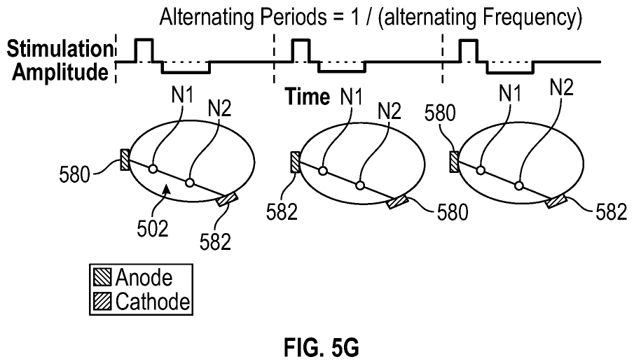

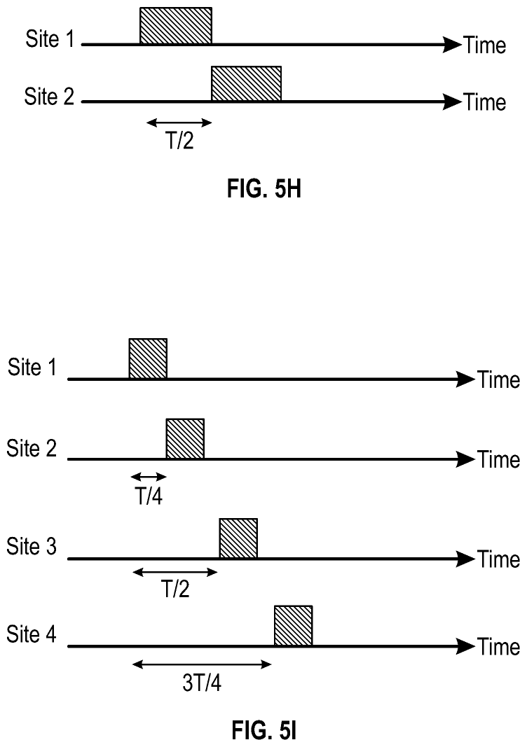

In some embodiments, the stimulation waveform is biphasic and charge balanced as shown in FIGS. 5A-5I. A biphasic waveform has a phase of a first polarity (e.g., positive current) and a phase of the opposite polarity (e.g., negative current). A charged balanced waveform has a net zero charge when the waveform is integrated over time (in other words, the cumulative area beneath the curve is zero). The stimulation waveform can have an excitatory phase/pulse and a charge balance phase/pulse with the opposite polarity. The excitatory phase may arise from an electrode serving as the excitatory electrode and the charge balance phase may arise from an electrode serving as the charge balance electrode. The excitatory electrode may be positioned over or proximate to one nerve (e.g., a first nerve) and the charge balance electrode may be positioned over or proximate to another nerve (e.g., a second nerve), as described elsewhere herein.

In some embodiments, the stimulation waveform excitatory phase can excite the nerve located near the cathode without exciting the nerve under the anode. In other embodiments, the excitatory phase may be an anodic phase while the charge balance phase may be the cathodic phase. In some embodiments, the excitatory and charge balance phases can have the same amplitude and duration, as shown in FIGS. 5A and 5B, with the stimulation amplitude as the Y axis and time as the X axis. The pulse width, pulse spacing, and period variables are also illustrated. This type of symmetrical stimulation waveform can tend to stimulate both nerves, such as simultaneously. FIG. 5A illustrates an embodiment of a biphasic stimulation waveform with a positive-going leading edge, while FIG. 5B illustrates an embodiment of a biphasic stimulation waveforms with a negative-going leading edge. In some embodiments as illustrated in FIGS. 5A-5B, the waveforms can have square or rectangular shapes with stimulation at maximum amplitude. Other embodiments can include curved waveforms where there can be a ramp-up and/or ramp-down period to or from maximum amplitude. Other embodiments can include a sinusoidal waveform.

FIG. 5C illustrates a schematic illustrating a waveform and a stimulation device 500 positioned on the skin of a subject's limb 502, shown in cross-section. The device 500 can include in some embodiments 3 electrodes to stimulate 2 nerves (nerves not shown) with the electrodes grouped as alternating pairs (pair 1 and pair 2). The electrodes serving as activated electrodes 504 and inactivated electrodes 506 during particular phases of stimulation are shown. In another embodiment, utilizing exactly two electrodes to stimulate exactly two nerves can utilize, for example, symmetrical stimulation waveforms such as shown, for example, in FIGS. 5A-5B to simultaneously stimulate exactly two nerves with exactly two electrodes.

In other embodiments, the excitatory and charge balance phases of the stimulation waveform can have different amplitudes and durations, yet still remain charge balanced or substantially charge balanced. For example, as shown in FIGS. 5D and 5E, a first stimulation waveform can have an excitatory phase with a greater amplitude but a shorter duration than the charge balance phase to stimulate the nerve close to the electrode serving as the excitatory electrode such that the excitatory phase and the charge balance phase are not symmetric (e.g., not mirror inverses of each other), with the stimulation amplitude as the Y axis and time as the X axis. The pulse width, pulse spacing, and period variables are also illustrated. This type of asymmetrical stimulation waveform can allow for alternating stimulation of nerves (e.g., only one nerve at a time in some cases). FIG. 5D illustrates an embodiment of a biphasic asymmetric stimulation waveform with a positive-going leading edge, while FIG. 5E illustrates an embodiment of a biphasic asymmetric stimulation waveforms with a negative-going leading edge. As shown, the asymmetric waveform can be configured to be charge balanced such that the area under the positive-going pulse 552 can be equal to the area under the negative-going pulse. In some embodiments as illustrated in FIGS. 5D-5E, the waveforms can have square or rectangular shapes with stimulation at maximum amplitude. Other embodiments can include curved waveforms where there can be a ramp-up and/or ramp-down period to or from maximum amplitude. Other embodiments can include sinusoidal waveforms.

In FIG. 5F the charge balance phase may initially have an amplitude that is equal to or greater than the excitatory phase, but its duration is relatively brief and the amplitude rapidly drops such that the waveform is not excitatory (the nerve is not stimulated), similar to a capacitive discharge. What is important in some cases is that the charge balance waveform is non-excitatory. The amplitudes and duration can be configured such that only one nerve, but not both nerves, is stimulated with the stimulation waveform, while the other (e.g., second) nerve is not stimulated by the stimulation waveform. A second stimulation waveform can also have an excitatory phase with a greater amplitude but a shorter duration than the charge balance phase, but the electrodes serving as the excitatory electrode and charge balance electrode can be switched, by reversing the polarities of the two electrodes, so that the second nerve is stimulated and the first nerve is not stimulated.

In some embodiments, the amplitude (e.g., the mean, median, or maximum amplitude) of the excitatory phase of the first stimulation waveform is about or at least about 2, 3, 4, 5, 6, 7, 8, 9, 10, 15, 20, or more times greater than the amplitude (e.g., the mean, median or maximum amplitude) of the charge balance phase of the first stimulation waveform, or within a range incorporating any two of the aforementioned values. In some embodiments, the pulse width can be between, for example, about 50 μs to about 1,000 μs. The stimulation amplitude can be, in some cases, between about 1-15 mA, or between about 1-10 mA, or between about 1-25 mA.

In some embodiments, the duration of the charge balance phase (either in total, or the duration of which the charge balance phase is at maximum amplitude or substantially at maximum amplitude) of the first stimulation waveform is about or at least about 1.25, 1.5, 2, 2.5, 3, 4, 5, 6, 7, 8, 9, 10, or more times greater than the duration of the charge balance phase of the first stimulation waveform, or within a range incorporating any two of the aforementioned values.

In some embodiments, as described above, the same two electrodes can switch functions as the excitatory electrode and charge balance electrode between stimulation waveforms/pulses so that each nerve can be stimulated serially (but not necessarily at the same time). In some implementations, this may be accomplished by switching the polarities of the two electrodes and applying the same or similar stimulation waveform from the second electrode as the first electrode, as illustrated schematically in FIG. 5G, showing exactly two electrodes for stimulating exactly two nerves (N1 and N2) on a patient's limb 502, and the electrodes serving as the anode 580 and the cathode 582 can change depending on the desired stimulation pattern. In other implementations, this may be accomplished by retaining the same polarities but by altering the amplitudes and durations of the cathodic and anodic phases, such that a cathodic electrode is used to stimulate the first nerve and an anodic electrode is used to stimulate the second nerve or vice-versa. In some embodiments, three, four, or more electrodes as part of an array, or multiple pairs of electrodes can alternate as either excitatory electrodes or charge balance electrodes as noted above.

Since the nerves can be independently stimulated, it allows for each nerve to be stimulated, in some embodiments at different times, as shown schematically in FIG. 5C above. For example, after the first nerve has been stimulated or has started being stimulated, the second nerve can begin stimulation after a delay that can be based on the period of the tremor, T, as shown in FIGS. 5H and 5I. For example, the delay can be the period of the tremor divided by the number of nerves to be stimulated, which can be exactly two when just the median and radial nerves are to be stimulated.

Some stimulation schemes can be designed to dephase, override or obscure an abnormal neural network. For example, in some embodiments for stimulation to reduce hand tremor, as illustrated in FIG. 5H, a conceptual diagram showing a sample excitation scheme to dephase brain regions receiving sensory input from two sites. For example, the two sites could be two locations on the wrist over the median and radial nerves. The stimulation at site 2 is delayed after site 1 by time T/2, where T is the period of the native tremor. For example, if the tremor is at 8 Hz the period is 125 ms and the stimulation of site 2 would be delayed by 62.5 ms. The stimulation is designed to reset the phase of the neuron, which may be implemented using high frequency stimulation (e.g., above 100 Hz) as shown in FIGS. 5H and 5I or a DC pulse. FIG. 5I is a conceptual diagram showing a sample excitation scheme to dephase brain regions receiving sensory input from four sites, with subsequent sites delayed by T/4. In some implementations, the stimulation scheme is periodic, repeating the stimulation pulses at regular intervals over a duration of time.

### D. Example Wearable Band

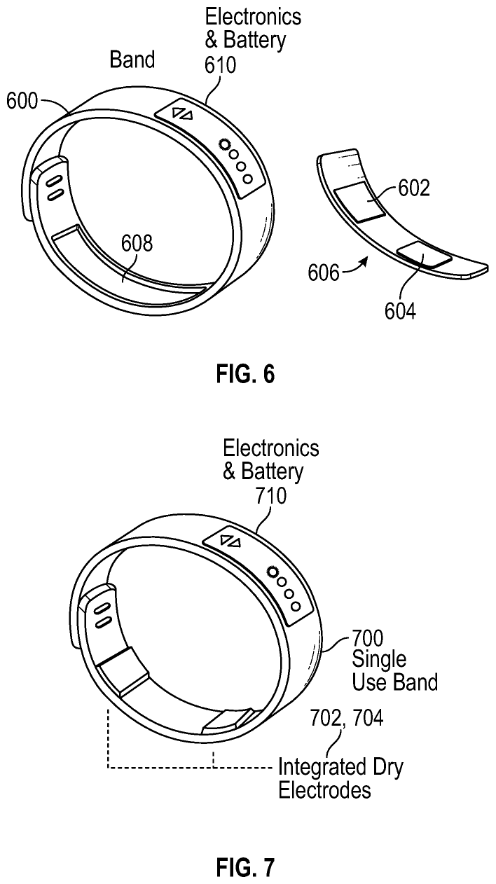

In some embodiments as shown in FIGS. 6 and 7, the electrodes can be disposed on a wearable band that can be worn around the wrist, arm, ankle, leg, or other limb or body part. The wearable band may include a removable/detachable controller as further described in International Application No. PCT/US2016/37080, titled SYSTEMS AND METHOD FOR PERIPHERAL NERVE STIMULATION TO TREAT TREMOR WITH DETACHABLE THERAPY AND MONITORING UNITS, which is hereby incorporated by reference in its entirety for all purposes. As shown in FIGS. 6 and 7, the wearable bands have two electrodes which can be used to stimulate up to two nerves. However, other embodiments can have N electrodes to stimulate up to N nerves, wherein N represents a variable integer number as described elsewhere herein.

FIG. 6 illustrates a wearable band 600 with disposable electrodes 602, 604. In some embodiments, the disposable electrodes 602, 604 can be coated or covered with an electrically conductive material, such as a solid hydrogel or a conductive liquid. The disposable electrodes 602, 604 may be disposed on a strip 606 that can be removably attached to the wearable band 600, which may have a receptacle 608 for receiving the strip 606. Other embodiments may comprise other means of attaching the strip 606 to the band 600. The strip 606 and the band 600 can have electrical contacts and a flexible circuit so that the electrodes are electrically connected to the controller 610. To accommodate various body part sizes, the disposable strip 606 can be provided with a variety of electrode spacings. This allows one band size, which can be adjustable (e.g., via an adjustable clasp or hook and loop fastener), to accommodate users with different body part sizes. In some embodiments, hydrogel coated electrodes may be more suitable for use with removable electrodes, as shown in FIG. 6, that can be disposed and replaced on a regular basis, such as every 1, 2, 3, 4, 5, 6, or 7 days, for example.

FIG. 7 shows a wearable band 700 with integrated electrodes 702, 704. In some embodiments, the integrated electrodes 702, 704 can be dry electrodes, described elsewhere herein, in electrical communication with a controller 710, comprising electronics for operating the device and a battery, which may be rechargeable. In some embodiments, the controller 710 may be detachable from the band 700. The electrodes 702, 704 may be in electrical communication with the controller 710 through a flexible circuit embedded in the band 700. Dry electrodes may be more suitable for longer term use electrodes that can be used for months, such as at least 3 months, before the band needs to be replaced. In some embodiments, the band may be a single use band that can be used for a relatively long period of time before replacement.

### E. Example Dry Electrodes

Hydrogel electrodes have two beneficial properties that provide uniform current distribution across the surface of the electrode, which improves comfortability of stimulation: (1) a water or gel based electrode surface allows for preferable conduction properties electrode, and (2) adhesion to the skin provides high skin conformance. This conformance and integrity of the contact can be important in some cases for comfortable electrical stimulation of sensory nerves below the skin surface.

However, the sticky hydrogel electrode can potentially provide challenges in usability for the wearer, as the hydrogel material may not allow movement (e.g., adjustment of a body-worn device), can be challenging to remove and apply (e.g., can lose its adhesive properties), can easily and quickly become dirty or degrade, especially in real-world environments, and can cause skin irritation. Thus, hydrogel electrodes may not be desirable for repeated, all day wear. For these reasons, it can be advantageous in some embodiments to develop a dry skin interface between the electrode and skin known as a “dry electrode” to deliver electrical stimulation, particularly for body-worn stimulation devices intended for long-term, repeated wear. It can also be challenging to develop a dry electrode material, because loading agents that allow for conduction also tend to increase material stiffness, which reduces conformance and leads to discomfort at the skin interface. Furthermore, it can in at least some cases be very difficult to manufacture dry electrodes which provide uniform field at the skin electrode interface. Examples of dry electrodes are described in International Application No. PCT/US2017/040920 (International Publication Number WO2018/009680) and U.S. Pat. No. 10,814,130, which are each incorporated by reference in their entireties.

An electrode for transcutaneous electrical stimulation and/or for electrical sensing can be used for many applications, including but not limited to peripheral nerve stimulation for treating tremor, osteoarthritis, overactive bladder, high blood pressure, dysrhythmias, pain, diabetes, and inflammatory diseases. Such an electrode may be a “dry” electrode or “wet” electrode. Dry electrodes advantageously do not require any adhesive or layer of conductive moisture (such as a gel or spray) to achieve the skin contact sufficient for comfortable delivery of electrical stimulation. In contrast, wet electrodes utilize either integrated adhesives or conductive gels and moisture to achieve that contact and electrical connection. Some such examples are hydrogels, which can be adhesive or non-adhesive. The gels and moisture tend to dry out over time and the adhesives tend to only be effective for one use due to contamination from adhesion of dead skin cells, dirt, etc. As such, wet electrodes tend to be not reusable or only reusable for a short period of time, for example less than one day, even when stored optimally. Dry electrodes allow the electrode to be effectively used for relatively long periods of times, such as for about or at least about 1, 2, 3, 4, 5, 6, 7, 8, 9, 10, 11, or 12 months, 1, 2, 3, 4, 5 years, or more before the electrode needs to be replaced.

There are several challenges in developing a dry electrode in a body-worn device that provides comfortable transcutaneous stimulation. First, the electrode can in some cases have a bulk resistivity (e.g., the inverse of conductance) that is near the resistivity of skin, or resistivity high enough to allow for a uniform distribution of current through the electrode, as concentration of current, especially around electrode edges or imperfections in the electrode surface, can cause uncomfortable stimulation. Most dry electrode materials utilize a polymeric base material, such as silicone, loaded with a conductive filler material, such as carbon. Bulk resistivity depends on the amount of filler material loaded into the base polymer, and also the resistivity of the base polymer. In some implementations, optimal bulk resistivity for providing good conduction in a body-worn device may be between about 25 and about 2000 ohm·cm, between about 50 and about 1000 ohm·cm, or between about 100 and about 420 ohm·cm.

Second, the electrode can be configured to be compliant enough to provide conformance to the skin, especially around bony structures such as the radius and ulnar bones in the wrist. If the material is too stiff and cannot conform to the skin, areas of the electrode surface can lift from the skin, causing concentrations in current and uncomfortable stimulation. Compliance of the electrode depends on the base polymer material properties, the amount of conductive filler material loaded into the base polymer, and the thickness of the electrode. For example, more filler material tends to lead to a less compliant (or stiffer) electrode. Additionally, a thicker electrode will tend to be stiffer than a thinner electrode. In some implementations, a preferred durometer for providing good conformance to a dry electrode may have a Shore hardness between about 25 A and about 55 A, between about 30 A and about 50 A, or between about 35 A and about 45 A.

Third, the electrode can in some cases have uniform material properties across the surface of the electrode, which must be controlled during manufacturing. Drastic inhomogeneities in properties of the electrode surface, such as resistivity or surface finish, can also cause concentrations in current and lead to uncomfortable stimulation. In some implementations, an optimal measure of homogeneity for providing uniform current distribution may be less than about 10%, 9%, 8%, 7%, 6%, 5%, 4%, 3%, 2%, or less difference in resistivity across the electrode surface. Uniform material properties of a dry electrode depend upon a uniform distribution of the conductive filler material in the base polymer and a uniform surface finish across the electrode, the latter of which is typically controlled by the surface finish of a mold. These three properties of the electrode affect each other, so designing a cost-efficient, manufacturable, and durable dry electrode material that provides comfortable transcutaneous stimulation requires optimizing these multiple parameters.

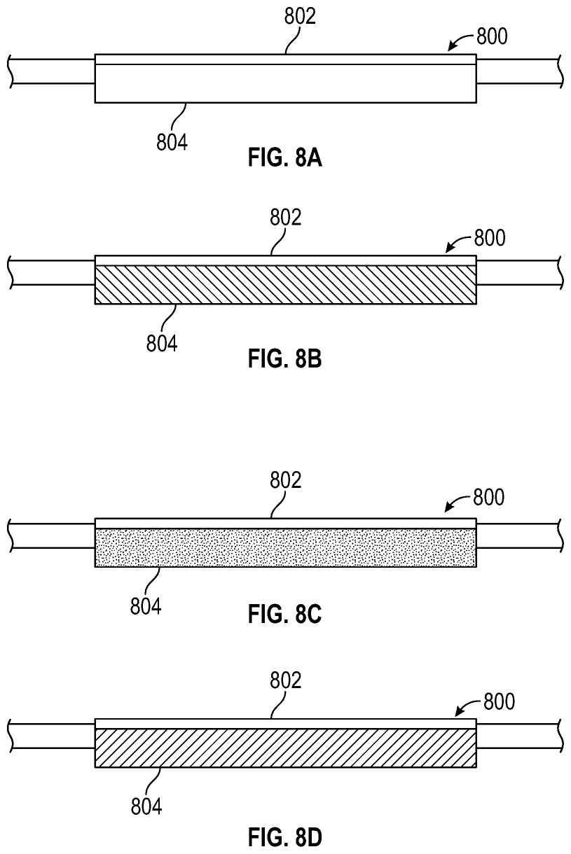

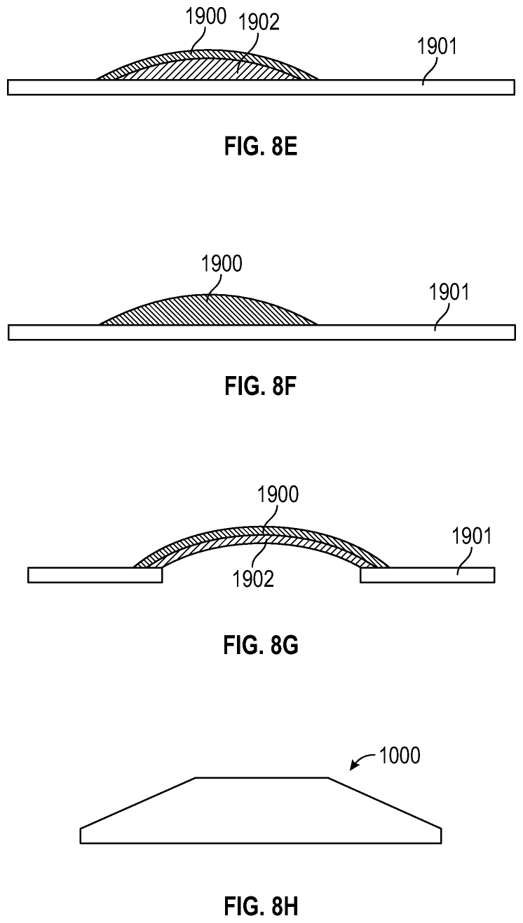

In some embodiments, as shown in FIGS. 8A-8D, the dry electrode 800 includes a conductive base layer 802 and a conductive skin contact layer 804 made of a conductive plastic, rubber, silicone material, or other suitable dry material. In some embodiments, the base layer 802 can include a thin layer of conductive material (e.g., copper, gold, silver-coated copper, stainless steel, silver, silver chloride, titanium, other metals or metal alloys, combinations thereof, polydimethylsiloxane, etc). In some embodiments, the base layer 802 can include metal coated polymer or plastic. In another configuration, the base layer 802 can include conductive polymer or plastic. In some embodiments, any two, three, or more materials such as those disclosed herein can be combined to make a base layer 802. The base layer 802 may be, for example, a continuous metal or a patterned metal. The size of metal patterns can be relatively large, such as, for example, between about 50% and about 70% of the surface area of the base layer 802, or about or at least about 50%, 55%, 60%, 65%, 70%, 75%, or 80% (or ranges incorporating any two of the aforementioned values) of the surface area of the base layer 802 and still maintain a uniformity of current density of the electrode depending on the conductivity of the dry electrode 800 as well as the frequency of the waveform being delivered. Keeping the base layer 802 thin may allow increased flexibility, desirable for body-worn applications, especially where a stiff material is used. Using a patterned metal may also enhance the flexibility of the base layer 802.

In some embodiments, the dry electrodes 800 need to maintain good skin contact across the surface of the skin contact layer 804 in order to deliver comfortable transcutaneous electrical stimulation at the appropriate current. As such, the materials that make up the dry electrodes 800 typically are more conformal materials that are loaded with conductive materials. Challenges also lie in the ability for the base layer 802 to maintain flexibility without deforming or fracturing. The strong, secure electrical connection between the base layer 802 and the conductive layer 804, which in some embodiments are two or more different materials, can also be important in some cases to prevent delamination, which can cause the conductance to greatly decrease.

In some embodiments, the skin contact layer 804 is disposed, layered or coated over the base layer 802 and may include any moldable polymer, rubber, or plastic, including but not limited to silicone, rubber, or thermoplastic urethane. The material could be loaded with one or more conductive fillers, including but not limited to metal, carbon (e.g., carbon nanotubes or carbon black), graphite, and metal coated particles (e.g., metal-coated, such as silver-coated glass microspheres or bubbles). In some embodiments, the skin contact layer 804 can include a thin layer of silver chloride.

In some embodiments, the conductive filler material may be a mixture of different filler materials with different conductivities. For example, in some embodiments, a first highly conductive material (e.g., a metallic material) may be mixed with a second material with lower conductivity with respect to the first conductive material, such as carbon black, in order to increase the conductivity of the second filler material. The second material may have better physical properties (e.g., reduced stiffness) than the first material. Mixing filler material may allow less filler material to be used, resulting in a more flexible skin contact layer 804.

In some embodiments, as shown in FIG. 8B, the conductive filler material can be in a fiber form. In some embodiments as shown in FIG. 8C, the conductive filler material can be in a powdered or particulate form when added to the silicone, rubber, or plastic material. In some embodiments, a powdered form may be preferable over a fiber form in order to create a more uniform conductance across the surface of the loaded material. Long fiber length materials may extend through the entire thickness of the skin contact layer 804 and/or may be more difficult to evenly disperse, thereby creating areas of high conductance in some locations and areas of low conductance where the fibers are absent. The areas of high conductance may transmit too much current to the skin and cause pain or discomfort. In contrast, powder materials may be more easily and more uniformly dispersed throughout the skin contact layer to yield a material with uniform conductance across its surface. However, in some embodiments, materials that include fibers can be utilized.

In some embodiments as shown in FIG. 8D, the skin contact layer 804 can include open cell foam treated with a conductive surface coating that causes the foam to be conductive. The open cell foam can provide a high surface area-to-volume ratio which allows increased incorporation of the conductive coating. In some embodiments, the foam pattern is random and in some embodiments, it is not random. In some embodiments, the skin contact layer 804 can include a non-conductive substrate made from a porous material, including but not limited to foam, neoprene, sponge, etc. filled with a conformal conductive coating that allows current to pass from the base layer backing 802 to the skin contact layer 804. In some embodiments, the skin contact layer 804 can include a thin layer of silver chloride.

In some embodiments, a fibrous filler material may tend to increase the stiffness or durometer of the loaded material more than a powdered filler material. Also, more generally, as more filler is added and the concentration of filler increases, the loaded material tends to increase in stiffness. However, a low durometer, flexible material can be desirable in some embodiments to result in good conformance to the skin, which improves the physical comfortability of wearing the electrode 800. In addition, poor conformance to the skin can lead in some cases to the concentration of current through the electrode 800 to a smaller area still in contact with the skin, leading to the perception of pain if the resulting current density is too high. Therefore, in some embodiments where increased flexibility is desirable, a powdered filler material may be preferred and the amount of filler material can be limited or reduced in order to keep the durometer of the material within a desired limit. In some embodiments, the powder or particulate filler material can possess a diameter, length, width, and/or thickness that is less than about ⅓, ¼, ⅕, 1/10, 1/100, or less than the thickness of the skin contact layer 804. The amount or loading of filler material can affect both conduction and stiffness. The optimal loading amount will generally depend on the materials used for both the skin contact layer 804 and the conductive filler. In embodiments having a skin contact layer 804 comprising a silicone base polymer material, silver-coated glass bubbles having diameters of, for example, between about 10 μm and about 100 μm, between about 18 μm and about 50 μm, or approximately 18 μm, 25 μm, 35 μm, or 50 μm. can have a loading for example, of between about 1% and about 40%, between about 3% and about 25%, between about 5% and about 20%, or about 5%, about 10%, or about 20% (measured by weight or volume).

In embodiments having a skin contact layer 804 comprising a silicone base polymer material, a preferred loading of conductive single walled carbon nanotubes (SWCNT) may be, in some embodiments, about or less than about 10%, 9%, 8%, 7%, 6%, 5%, 4%, 3%, 2%, 1%, or less, or between about 1% and about 5% (measured by weight or volume). In some embodiments, incorporation of such materials can be unexpectedly advantageous when used as dry electrode materials. For example, glass bubbles can reduce weight of material, as the density is lower than most polymers (because bubbles are filled with air). Conductivity of the filler material can be controlled with the type of silver or silver alloy applied to the glass bubble. The spherical shape of the bubbles can advantageously allow for uniform or substantially uniform dispersion the in base polymer. SWCNTs have high conductivity (e.g., 10⁶ to 10⁷ S/m) and robust mechanical properties (combination of stiffness, strength, and tenacity) as a filler material within a polymer. Loading of SWCNTs can be more efficient (by weight or volume) than carbon black or carbon fibers as the structure of SWCNTs allow better transfer of their mechanical load to the polymer matrix. Conductivity of SWCNTs can be controlled during manufacturing by the chiral vector, C=(n, m), the parameter that indicates how the graphene sheet is rolled to form a carbon nanotube. Both materials can advantageously require less loading by volume to achieve required conductivity and changes in conductivity are less sensitive to deformation of the electrode material, for example due to applied pressure or forces during wear.

In some embodiments, any number of the following materials could be incorporated into a dry electrode: metals and metal alloys (e.g., stainless steel, titanium (e.g., 6Al-4V (Ti64) or cobalt chrome); graphite coated with pyrolytic carbon (e.g., pyrolytic carbon having 5 μm particle size, 0.8 μm pore size, and 20% Volume total porosity); a conductive ink/coating (e.g., silver or silver chloride printed ink); a self-wound transfer adhesive that can include an electrically conductive pressure-sensitive adhesive, (e.g., a 33 μm thick EC-2 electrically conductive acrylic); a double-sided, isotropically conductive pressure sensitive tape which conducts electricity through the thickness (the Z-axis) and the plane of the adhesive (X, Y planes), for example silicone electrically conductive adhesive transfer tape; a conductive fabric having multiple layers (e.g., screen printed breathable fabric electrode arrays); a textile electrode that includes silver-coated nylon (e.g., a conductive fabric that includes silvered polyamide and/or Spandex); silver-plated, aluminum-filled fluorosilicone; a thermoplastic elastomer with conductive particle filler; silicone filled with nanoparticles (e.g., silver nanoparticles; thermoplastic polyolefin elastomer (TEO), and/or thermoplastic vulcanizate (TPV) styrene ethylene butylene styrene (SEBS) alloy with carbon black electrically conductive silicone (e.g., silver plated aluminum silicone elastomers with silver fillers; conductive elastomers such as silicone with carbon black, for example a two-part, black, static dissipative silicone exhibiting rheology thixotropic behavior and/or a hydrogel, for example a 0.8 mm thick crosslinked hydrophilic polymer designed for stimulation applications.

The resistance of the skin contact layer 804 can increase proportionally with thickness; therefore minimizing thickness of the skin contact layer 804 can improve conductivity of the electrode 800. Higher resistance in the electrode 800 due to a thicker skin contact layer 804 could increase power required in the system to maintain the desired current. However, if the skin contact layer 804 is too thin, variations in the processing could cause significant inhomogeneity in the material properties and/or conductance at the skin contact layer 804. The skin contact layer 804 thickness can be, in some embodiments, between about 0.25 mm and about 5 mm, between about 0.5 mm and about 2 mm, between about 0.5 mm and about 1 mm, between about 1 mm and about 2 mm, between about 0.15 mm and about 10 mm, or ranges including any two of the aforementioned values or ranges there between.

Overall, the thickness of the electrode 800 can affect its flexibility and stiffness, with the stiffness increasing with increasing thickness. In some embodiments, the thickness of the skin contact layer 804 depends of the material properties (e.g., natural durometer) of the selected material, such as silicone, the choice of filler (e.g., for providing conductivity), and the desired durometer and desired resistivity or conductance of the conductive filler loaded skin contact layer 804. In some embodiments, the skin contact layer 804 of the electrode 800 can have a durometer between a shore hardness of about 10 A to 50 A, between about 10 A and about 30 A, between about 50 OO and about 50 A, between about 40 OO and about 70 A, or ranges including any two of the aforementioned values or ranges there between. In some implementations, these durometers can provide good conformance to the skin. As discussed above, the durometer can be controlled by various factors, including material selection, thickness of the layers, and the type and/or amount of the conductive filler added to the skin contact layer 804.

Additionally, the surface tackiness of the electrode 800 could be modified to enhance or decrease gripping against the skin (i.e., friction or resistance to shear force). Enhanced gripping (as in the case of a fully smooth electrode) could aid in skin conformance and reduction of sliding, which would reduce the unpleasant changes in stimulation intensity experienced by a wearer. In some embodiments, the skin-contacting surface 804 of the dry electrode 800 is smooth and flat, and lacks any spikes, projections, bumps, microneedles, or similar features. However, other embodiments could have a curved, domed, or tapered shaped electrode surface that could help improve contact with the skin at the center of the electrode, where current is delivered, by applying more contact force and reducing likelihood of sliding.

In some embodiments, the curved or domed electrode 1900 can have a backing material 1902 that is as or more compliant as the electrode material 1900, as illustrated in FIG. 8E, to apply appropriate pressure for good skin conformance. Also illustrated is a band 1901 which can be circumferential in some embodiments and configured to attach to a plurality of spaced apart electrodes as disclosed elsewhere herein. In some embodiments, the curved dry electrode 1900 does not require a separate backing material, as illustrated in FIG. 8F. In some embodiments, as illustrated in FIG. 8G the backing material 1902 is also curved or domed to match the shape of the dry electrode 1900, which could provide beneficial adhesion of the two layers during manufacturing or control stiffness of the dry electrode assembly. FIG. 8H schematically shows a side view of a tapered electrode 1000 to improve the conformability and control of delivery of current at the edges of the electrode. As shown, the electrode 1000 can be thicker in the center of the electrode than at the peripheral edges of the electrode. In some embodiments, the thickness at the center of the electrode 1000 can be about or at least about 1.5×, 2×, 2.5×, 3×, 4×, 5×, 6×, 7×, 8×, 9×, 10×, or more times the thickness at the peripheral edges of the electrode.

Additionally, concentrated delivery of current to the skin due to edges of the electrode can in some cases cause pain or discomfort; a curved shape would also increase the radius of edges to reduce likelihood of current concentrations. However, other embodiments can include one or more of the aforementioned features. However, if too tacky, this could make the band of a wearable device difficult to slide over the appendage. In this case, more subtly texturing the surface and/or treating the skin contact layer 104 with a coating could provide a more moderate level of tackiness.

The thickness and patterning of the base layer 1002 can also in some cases affect the ability of the electrode to mechanically bend and conform to different body parts, like the arm, wrist, hand, knee, ankle, or leg, for example. Continuous metal foils or films on thin polyimide substrates can be good candidates for producing flexible bands. Continuous metals can, in some embodiments, preferably be ductile enough to produce the bends needed for the particular appendage—for instance in a typical gold coated flex application, copper is coated with nickel and then passivated with gold. In some embodiments, because nickel tends to be brittle and crack, only a thin layer of nickel is desired. This flexibility could also be increased by patterning the backing material in a serpentine fashion along the direction of the bending. Additionally, other more ductile metals such as silver could be used for the conductive base layer backing 802.

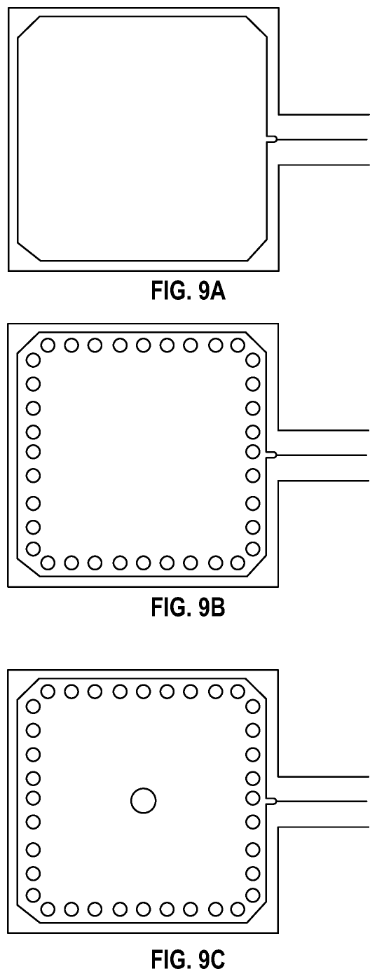

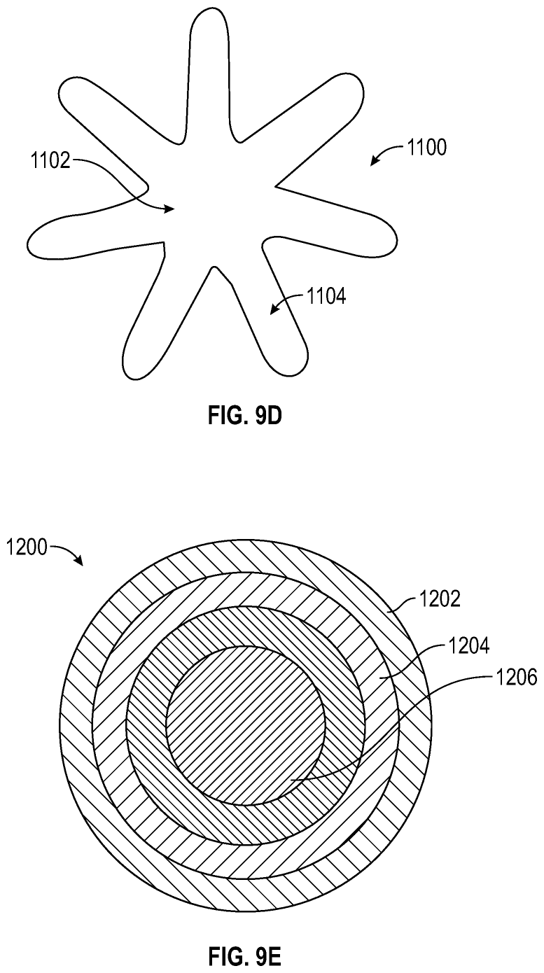

In some embodiments, a reduction in the adhesion of the skin contact layer to the base layer (e.g., delamination) can inhibit the electrical current from passing to the wearer. In some embodiments, adhesion can be improved using mechanical elements, as shown in FIGS. 9A-9C. FIG. 9A illustrates a base layer 1002 with no mechanical attachment or adhesion means for adhering to the skin contact layer. FIG. 9B illustrates a base layer 1002 with a plurality of holes 1003 positioned uniformly around the perimeter of the base layer 1002. FIG. 9C illustrates a base layer 1002 with a plurality of holes 1003 positioned uniformly around the perimeter and a larger hole 1003′ positioned approximately in the center of the base layer 1002. These holes 1003, 1003′ allow the skin contact layer material (e.g., silicone) to surround (e.g., penetrate) the base layer 1002 upon application (e.g., over-molding) and maintain good contact. Any combination of these holes 1003, 1003′ may be used across the base layer 1002. In addition, surface treatments such as primers, corona or plasma treatments can be used to prep the surface of the base layer 1002 material for promoting adhesion of the skin contact layer.

In some embodiments, instead of over-molding directly onto the base layer, the skin contact layer can be adhered to the base layer using loaded glues (e.g., silver epoxies) or other conductive adhesives such as Z-axis tapes that allow conduction only in the z-direction across the thickness of the tape and not along the length or width of the tape (i.e., the direction perpendicular to the surface of adhesion). Provided the interface is thin enough, nonconductive adhesives can also be utilized in some embodiments.

To provide stimulation that is comfortable to the wearer, several features of the electrode can be desirable in some embodiments as discussed elsewhere herein. One electrode feature in some embodiments is to provide the electrode surface with a substantially uniform homogeneity of conductance, meaning that the current is transmitted evenly across the skin contact surface of the electrode. To validate whether the electrode surface has a substantial uniform homogeneity of conductance, the end-to-end resistance or conductance of the skin contact layer, or the entire electrode with the base layer added to the skin contact layer, can be measured at a plurality of points across the entire skin contact surface of the electrode. In some embodiments, the standard deviation of the measured resistivity or conductance at the plurality of points is less than about 10, 15, 20, 25, 30, 35, 40, 45, or 50 (on an absolute, like ohm-cm, or percentage basis) more or less than the average value or mean value of the measured resistivity or conductance. In some embodiments, verifying whether the current passing through the skin contact surface is uniform can be measured at a plurality of points across the entire skin contact surface of the electrode. In some embodiments, the standard deviation of the measured current at the plurality of points is less than about 10, 15, 20, 25, 30, 35, 40, 45, or 50 (on an absolute, like milliamps, or percentage basis) more or less than the average value or mean value of the measured current.

In some embodiments, the dry electrodes can be disposed in a band that applies pressure, such as radially inward pressure in some cases, in order to maintain good contact and a good electrical connection with the skin. Various embodiments of the band, such as a D-ring, or inflatable cuff, are all bands that when combined with thin dry electrodes can provide the skin conformance needed to provide good electrical contact. In some cases, the pressure required to provide effective electrical contact is around 10-40 mmHg, around 5-50 mmHg, around 15-300 mmHg, or ranges including any two of the aforementioned values or ranges there between.

In some embodiments, the conductive material forming the skin contact layer has a high volume resistivity that can be between about 1 ohm·cm and about 2000 ohm·cm, between about 20 ohm·cm and about 200 ohm·cm, between about 100 ohm·cm and about 1000 ohm·cm, between about 5 ohm·cm and about 100 ohm·cm, between about 1 ohm·cm and about 10,000 ohm·cm, or ranges including any two of the aforementioned values or ranges there between. These resistivity ranges can be comparable with current hydrogel electrodes. A lower volume resistivity can result in discomfort during stimulation, while a higher volume resistivity can result in power loss. Therefore, in some embodiments, a moderate volume resistivity may be optimal.

In some embodiments, the electrode can have a lower resistivity towards the center of the electrode and a higher resistivity toward the edges of the electrode to reduce the likelihood of concentration of current being delivered through the edges of the electrode (i.e., spreading the current across the surface of the electrode). Concentrated delivery of current to the skin due to edges of the electrode can cause pain or discomfort in some embodiments. Resistivity of the electrode can be controlled by varying the concentration of conductive filler material throughout the electrode 1200, as illustrated in FIG. 9E, schematically illustrating a cross-section of a dry electrode that can include a first outer zone 1202 with less filler material at the edge of the electrode with relatively low conductivity, a second middle zone 1204 with more filler material than the outer zone 1202 with relatively intermediate conductivity, and a third inner zone 1206 with more filler material than the first outer zone 1202 and the second middle zone 1204 and relatively high conductivity. Some embodiments could have a different number of zones, such as two, four, five, or more zones, or a gradual conductivity gradient within each zone.

In some embodiments, the resistivity of the electrode can be controlled by varying the profile shape of the electrode, such as a star pattern 1100 as illustrated in FIG. 9D, or by varying the thickness of the electrode, such as having an electrode that is thicker toward the center and thinner toward the edges, as illustrated in FIG. 8E, which illustrated the side view of an electrode with a tapered profile. FIG. 8E schematically shows a side view of a tapered electrode 1000 to improve the conformability and control of delivery of current at the edges of the electrode. As shown, the electrode 1000 can be thicker in the center of the electrode than at the peripheral edges of the electrode. In some embodiments, the thickness at the center of the electrode 1000 can be about or at least about 1.5×, 2×, 2.5×, 3×, 4×, 5×, 6×, 7×, 8×, 9×, 100×, or more times the thickness at the peripheral edges of the electrode. As illustrated in FIG. 9D, the top view of the star pattern electrode 1100 can include a central hub 1102 with a plurality, such as about or at least about 3, 4, 5, 6, 7, 8, 9, 10, or more radially outwardly extending projections 1104. The electrode can be integrally formed in some embodiments. In some embodiments, the patterned electrode can include a non-conductive material, such as silicone, wherein the star or other pattern is conductive and the outer, surrounding material is non-conductive, such as via a two-part molding process. In some embodiments, controlling the spatial resistivity of the electrode, for example by the amount of filler material, can also control the location of current delivery to stimulate skin and nerves in one or more preferred locations.

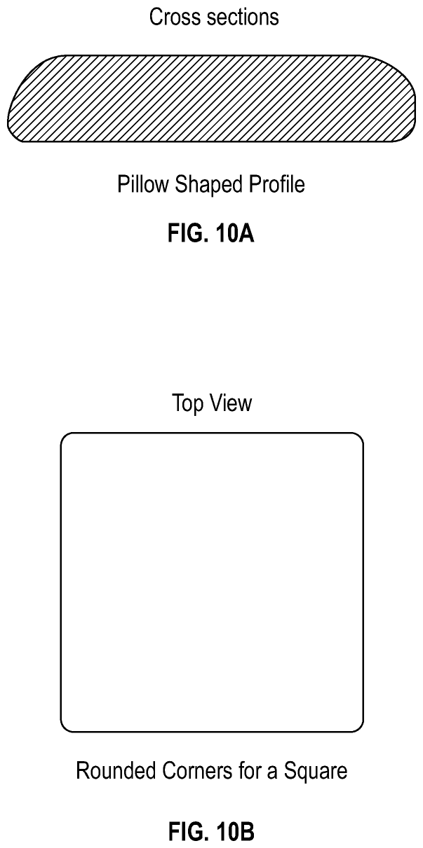

The shape of the electrode can also contribute to the ability to conform to the skin. If an electrode has edges and/or corners, such as in a square or rectangle shape, for instance, the edges and corners can deform and bend downward into the wearer's skin. This deformation can lead to uneven pressure at the skin contact layer, which can lead to localized concentration of current flow and discomfort. FIGS. 10A and 10B illustrate electrode shapes that may optimize conformity with the skin and homogenous current distribution. FIG. 10A illustrates a side cross sectional view of an electrode 1100. FIG. 10B illustrates a top view of an electrode 1100′, which may be the same or a different electrode as electrode 1100 shown in FIG. 10A. Shapes, such as the pillowed-shape profile electrodes with tapered (e.g., rounded, non-sharp) edges as illustrated in FIG. 10A and/or electrodes with rounded corners as illustrated in FIG. 10B, prevent discomfort by reducing deformation of the electrode around the corners and edges when coming into contact with a person's skin. In some embodiments, the electrodes can have a generally square or rectangular geometry. In some embodiments, the electrodes can have an arcuate, e.g., circular or oval geometry (e.g., from a top view). Additionally, electrodes can in some cases protrude beyond the band surface to ensure that the electrode is the primary material touching the person's skin. Additionally, the shape of the electrode can also help with conformance—for instance, squares with rounded corners can reduce the chances of a single point contacting the skin.

### F. Example Self-Wetting Material for Electrodes

As discussed above, the electrode may be a wet electrode or a dry electrode. Although a dry electrode may not require any adhesive or layer of conductive moisture (such as a gel or spray) to achieve the skin contact sufficient for comfortable delivery of electrical stimulation, the dry electrode used for the skin interface may need a wetting agent in some situations to ensure a low resistance connection between the electrode and surface. In some embodiments, the signal quality or treatment efficiency may be better if a wetting agent is between the dry electrode and skin. On the other hand, a wet electrode which utilizes either integrated adhesives or conductive gels and moisture to achieve the contact and electrical connection may dry out over time.

In some embodiments, a wetting agent may be applied to the skin where the electrodes are to be placed before placing the apparatus to the skin to ensure better signal quality and/or adequate electrical conductivity between the electrodes and skin to comfortably deliver electrical current during a stimulation session. In some embodiments, the wetting agent may include, but is not limited be a water, gel, salt water (e.g., sweat). In some embodiments, the wetting agent may evaporate over the course of a stimulation or monitoring session, which may potentially increase the risk of a patient's discomfort or skin irritations. Thus, in some situations, users may be forced to wet their skin several times during a treatment or sensing period as the length of therapy or wear time is increased, which may cause the frustrations to users.

In some embodiments, antistatic material may be added to a dry or wet electrode material to provide self-wetting capability. The antistatic material may include migratory additives that are incompatible with silicone. The antistatic material may have a partially hydrophilic structure and may react with or attract moisture, which creates conductive pathways and/or lowers the resistivity of the part and allows excess electrons to dissipate.

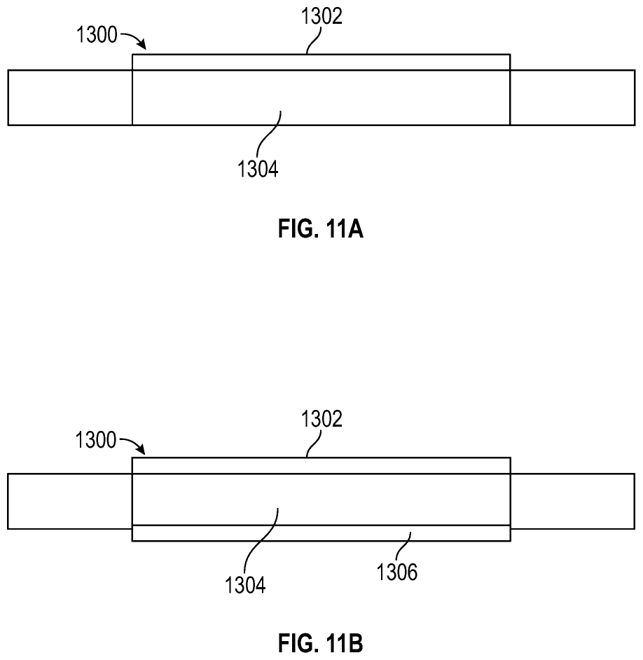

FIGS. 11A-11B schematically illustrate an example embodiment of an electrode before and after the electrode is placed on the skin of a user respectively. In some embodiments, the electrode 1300 in FIGS. 11A-11B may be a dry electrode and/or wet electrode. FIG. 11A shows an electrode 1300 comprising a base layer 1302 and a skin contact layer 1304 before being placed in contact with a user's skin or other heat source. The skin contact layer 1304 may comprise antistatic material and silicone. FIG. 11B illustrates the electrode 1300 after being placed on the user's skin or placed in contact with other heat source. As the skin contact layer 1304 of an electrode 1300 may comprise silicone, the antistatic material may bloom to the surface of the skin contact layer 1304 of the electrode 1300 and form a non-sticky, moisturizing, and conductive lubricant 1306 after being heated to a certain temperature. In some examples, the dry electrode 1300 may be heated to a certain temperature by body temperature when being attached to a patient's skin for a certain period of time. In some embodiments, the antistatic material may stop blooming to the surface of the skin contact layer 1304 after the antistatic material substantially covers the surface of the skin contact layer 1304.

In some embodiments, the antistatic material may have Generally Recognized as Safe (GRAS) certification granted by Food and Drug Administration (FDA), and may be for example, RD Abbott's antistatic material. In some embodiments, the antistatic material is a coconut oil derivative.

In some embodiments, the antistatic material may be added to skin contact layer of the electrode as a medium or heavy amount without impact to the properties of the electrode material. The amount of antistatic material in the formulation may directly impact the length of wear. The amount of antistatic material used in the formulation may depend, at least partially, on the length of use and/or the amount that needs to bloom to the surface to provide sufficient moisturizing and conductive lubricant.

In some embodiments, reducing a system impedance between the self-wetting electrode(s) 102 and the skin can lower the level of the stimulation amplitude necessary for paresthesia sensations during therapy. In some embodiments, the neurostimulation device 100 determines an impedance of the self-wetting electrode 102 and activates the neurostimulation device 100 to deliver electrical current through the self-wetting electrode 102 at least in part based on the measured impedance. In some embodiments, the impedance is based at least in part on a relative conductance between the self-wetting electrode 102 and the skin of the user.

As discussed in the section above titled E. Example Dry Electrodes, an electrode may be configured to be compliant enough to provide conformance to the skin, especially around bony structures such as the radius and ulnar bones in the wrist. If the material is too stiff and cannot conform to the skin, areas of the electrode surface can lift from the skin, causing concentrations in current and uncomfortable stimulation. In some implementations, a preferred durometer for providing good conformance to a dry electrode with antistatic materials may have a Shore hardness between about 25 A and about 70 A, between about 30 A and about 65 A, between about 35 A and about 65 A, between about 35 A and about 50 A, or between about 35 A and about 45 A, without impacting the materials in the dry electrode, such as the nanotubes. In some embodiments, alternative durometers of dry electrode material, such as silicone, may be possible to leverage the material across more anatomies to allow a longer wear time.

### G. Example Active and Passive Wetting Structures and Methods for Electrodes

In some embodiments, the surface of skin contacting layer of an electrode may be actively wetted by a wetting agent. In some embodiments, the wetting agent may be stored in a reservoir before wetting the skin contacting surface of the electrode. In some embodiments, the wetting agent may include, but is not limited to, water, electrolyte fluid or gel, salt solvent, and/or sweat of a user. In some embodiments, the reservoir may be channels or chambers. In some embodiments, the patient's sweat may be captured by the reservoir and used as a wetting agent. In some embodiments, the reservoir may be pre-filled with wetting agent or the user may fill or re-fill the reservoir with wetting agent. In some embodiments, the wetting agent may be a capacitance lowering agent which may ensure better electrical conductivity between the electrode and the skin of the user.

In some embodiments, active wetting of the surface of the skin contacting layer of an electrode may require the movement of the wetting agent between the patient's skin and stimulating electrode surface. In some embodiments, the slow transfer of the wetting agent may be achieved by, for example, using capillary action, osmosis, or micropump. In some embodiments, the transfer of the wetting agent may be passive. In some embodiments, a user may intentionally activate the transfer of the wetting agent from the reservoir to the area between the electrode and user's skin by methods, such as electronic methods and/or mechanical pushing on the band to force the wetting agent to be delivered between the electrode and the skin.

In some embodiments, a passive method may be utilized to wet the skin contacting surface of the electrode. In a passive method, the moisture and sweat that may lower surface capacitance may be retained passively. In some embodiments, micro-sized structures may be molded into the electrodes and/or bands and may serve as sweat reservoirs. In some examples, the micro-sized structures may include, but not limited to, microchannels, grooves, or ridges. The captured sweat may help maintain a sufficient conductivity between the electrode and user's skin.

In some embodiments, both active wetting and passive wetting methods may be used to improve the conductivity between the skin and the electrode. In some embodiments, the active and/or passive wetting methods may be used together with electrode comprising the antistatic materials as discussed above.

### H. Example Transcutaneous Stimulation Induced Self-Wetting

In some embodiments, a patient's sweat may be induced by delivering a transcutaneous stimulation to the patient's median, radial, or ulnar nerves at the forearm, wrist, and/or fingers. The resultant sweat is highly conductive and will naturally accumulate at the electrode-skin interface. Such induced sweat may be used as a wetting agent and help maintain sufficient electrode-skin conductivity with non-invasive, transcutaneous electrical stimulation.

It was previously shown that stimulation delivered percutaneously to the median nerve at the wrist with microneedles may activate sweat glands and increase sweating at the ipsilateral palm and fingers. Further details are described, for example, in a paper titled “Neuroeffector characteristics of sweat glands in the human hand activated by regular neural stimuli” by M. Kunimoto et al., which is incorporated herein by reference in its entirety. For transcutaneous stimulation, the amplitude may need to be sufficiently high to activate C-type sudomotor neurons that are in the target nerve(s) and that innervate downstream (anterograde) sweat glands.

In some embodiments, the amplitude of the transcutaneous stimulation may depend, at least partially, on the patient and the placement of electrodes. In some embodiments, the amplitude may be about 1 mA to about 10 mA. In some embodiments, the frequency of the transcutaneous stimulation for sweat induction may be about 15 Hz, about 20 Hz, about 25 Hz, about 30 Hz, or higher. In some embodiments, the pulse width of the transcutaneous stimulation may be about 100 microseconds, about 150 microseconds, about 200 microseconds, about 250 microseconds, or any other time length. In some embodiments, to maximize the degree of sweating, the stimulation may have a frequency of about 20 Hz or higher and/or a pulse width of about 150 to about 250 microseconds.

As discussed previously, in some embodiments, the electrodes are used in a device and system that provides peripheral nerve stimulation, targeting individual nerves, to provide therapeutic benefit across a variety of diseases, including but not limited to movement disorders (including but not limited to essential tremor, Parkinson's tremor, orthostatic tremor, and multiple sclerosis), urological disorders, gastrointestinal disorders, cardiac diseases, inflammatory diseases (for example neuroinflammation), mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy is treated in one embodiment. Inflammatory skin conditions and immune dysfunction are also treated in some embodiments. In some embodiments, provided herein are treatments of restless leg syndrome, periodic limb movement disorder, repetitive movements of the limbs and abnormal sensation. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s).

In some embodiments, the stimulation parameters for inducing sweat may be different from the parameters for delivering a main therapy session for the above-mentioned various diseases. In some embodiments, the stimulation for the main therapy session of the various diseases may be at high frequency (e.g., above 100 Hz). In some embodiments, the frequency of stimulation for the main therapy session of the various diseases, such as essential tremor, may be about 100 Hz, about 125 Hz, about 150 Hz, about 175 Hz, about 200 Hz, or higher. In some embodiments, the pulse width of stimulation for the main therapy session of the various diseases, such as essential tremor, may be from about 50 microseconds to about 1000 microseconds, such as about 250 microsecond, about 300 microsecond, about 350 microsecond, or longer period of time. Although frequencies below 5 kHz are used in several embodiments, some embodiments use higher frequency stimulation (e.g., of nerves at or near the wrist or ear) of 5-75 kHz (e.g., 10-40 kHz, 15-60 KHz, etc.) and a pulse width of 1-20, 10-50, 10-40 microseconds.

In some embodiments, the amplitude of the stimulation for therapy of various diseases, such as essential tremor, may be about 1 mA to about 25 mA, about 1 to about 15 mA, about 1 to about 10 mA. In some embodiments, the amplitude of the stimulation needed to activate Aβ-type nerve fibers may be lower.

In the situation where at least one of the parameters of the stimulation for main therapy session of various diseases, such as essential tremor, is different from the parameters of the stimulation for sweat induction, the system and device may periodically alternate between delivering stimulation with parameters for the main therapy session and parameters for sweat induction. In some embodiments, the time for main therapy session may be longer than the time for sweat induction session. In some embodiments, the ratio of time for main therapy session and time for sweat induction session may be about 50, about 40, about 20, about 10, about 5, or any other ratio. In some embodiments, the system and device could alternate between about 1 minute of parameters for sweat induction and about 40 minutes of parameters for the main therapy session. In some embodiments, the system and device may alternate between about 2 minutes of parameters for sweat induction and about 40 minutes of parameters for the main therapy session. In some embodiments, the system and device may alternate between about 4 minutes of parameters for sweat induction and about 40 minutes of parameters for the main therapy session. Thus, a user may not need to wet their wrist with a wetting agent before placing the band and/or electrode on the skin.

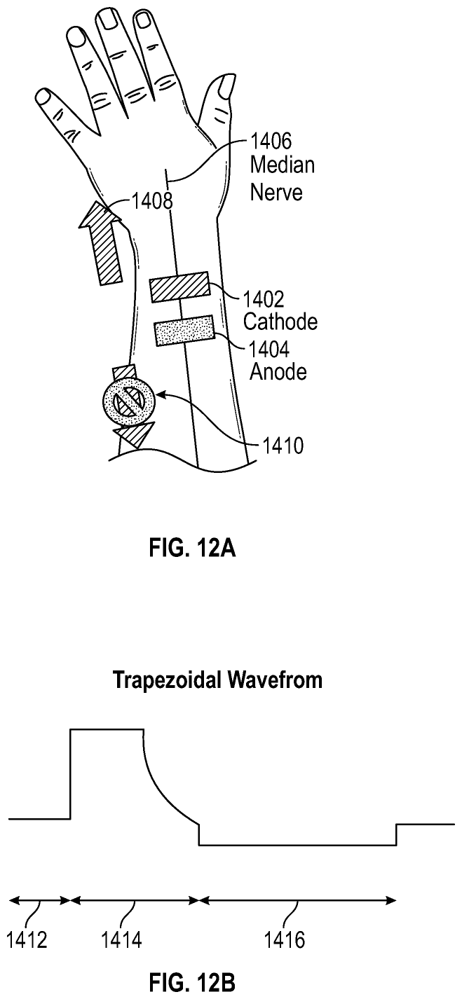

In some embodiments, the stimulation for sweat induction may cause patient discomfort by activating Aδ and C-type pain fibers. In some embodiments, a bipolar electrode configuration with proximal anode and distal cathode may be used in conjunction with a trapezoidal waveform to block the resultant afferent pain signals. FIG. 12A illustrates such a configuration and placement of the cathode and anode and FIG. 12B is a schematic illustration of trapezoidal waveform according to some embodiments.

As shown in FIG. 12A, a distal cathode 1402 and a proximal anode 1404 are longitudinally placed along the target nerve on the same side of the arm relative to the target nerve. In some embodiments, the distal cathode 1402 and the proximal anode 1404 are spaced from each other by a distance, for example, 2, 3, 4, 5, 6, 7, 8, 9, 10 mm or more or less, or ranges incorporating any two of the foregoing values, for example, about 3 to 9 mm, about 5 to 7 mm, or ranges there between.

In some embodiments, the target nerve may be a median, ulnar, and/or radial nerve. The cathode may be the negative pole because it discharges anions, and the anode may be the positive pole because it discharges cations in a stimulator. By placing the cathode 1402 proximal and the anode 1404 distal, the stimulation is delivered in direction 1408, rather than direction 1410. Placement of proximal cathode and distal anode may cause anodal blocking and the stimulation may be propagated in only one direction 1408. Thus, the Aδ and C-type pain fibers may not be activated and/or the pain signals may be blocked.

FIG. 12B is a schematic illustration of an example waveform (e.g., trapezoidal waveform). There may be two phases in a trapezoidal waveform: an excitatory phase 1414 and a negative charge phase 1416. There may also be a neutral phase spacing 1412 between the excitatory phase 1414 and a negative charge phase 1416. In an excitatory phase 1414, the amplitude may maintain at a maximum for a period of time and then decreases gradually at the end of the excitatory phase 1414. The gradual decrease at the end of the excitatory phase 1414 may help to suppress ‘anode break’ excitation, which can otherwise occur on nerve fibers near the anode at the end of the excitatory phase 1414 if the excitatory phase ends abruptly. In some embodiments, the maximum, mean, and/or median amplitude of the excitatory phase 1414 may be about 1 mA to about 10 mA. In some embodiments, the maximum, mean and/or median amplitude may depend, at least partially, on the amplitude to induce sweat. In some embodiments, the maximum, mean and/or median amplitude may be higher than the amplitude to induce sweat. In some embodiments, the time constant of the waveform can be in the range of about 350 and about 1000 microseconds, between about 350 and about 750 microseconds, between about 350 and about 500 microseconds, between about 500 and about 1000 microseconds, between about 500 and about 750 microseconds, or ranges including any two of the aforementioned values or ranges there between.

In some embodiments, a negative charge phase 1416 may follow the excitatory phase 1414. In some embodiments, the negative charge phase 1416 may be longer than an excitatory phase 1414. In some embodiments, the length of the negative charge phase 1416 may depend, at least partially, on the time needed to balance the charge in the excitatory phase 1414. In some embodiments, the maximum, median, and/or mean amplitude of the negative charge phase 1416 may be less than about 10 mA. In some embodiments, the maximum, median, and/or mean amplitude of the negative charge phase 1416 may be, at least partially, related to the activation threshold of the Aδ and/or C-type pain fibers. In some embodiments, the maximum, mean, and/or median amplitude of the negative charge phase 1414 may not activate the Aδ and/or C-type pain fibers. In some embodiments, the maximum, mean, and/or median amplitude of the negative charge phase 1414 may be less than the maximum mean, and/or median amplitude of the excitatory phase 1414.

In some embodiments, the frequency of the excitatory phase 1414 may be about 15 Hz, about 20 Hz, about 25 Hz, about 30 Hz, or higher. In some embodiments the pulse width of the excitatory phase 1414 may be about 100 microseconds, about 150 microseconds, about 200 microseconds, about 250 microseconds, or any other time length. The combination of the electrode placement and trapezoidal waveform illustrated in FIGS. 12A and 12B may block the resultant afferent pain signals.

In some embodiments, a resistive heating element and/or thermoelectric cooling element may be placed on the forearm, wrist, or fingers to safely increase skin temperature, as a means to naturally induce sweating via temperature differentials. The temperature may be configured to not cause discomfort to the user but induce sweat to the user. In some embodiments, the resistive heating element and/or thermoelectric cooling element may be used together with the stimulation to induce sweat, and/or other means to wet the contact area between the electrode and skin.

The neuromodulation devices e.g., neurostimulation devices, described herein, in several embodiments, can be used for the treatment of depression (including but not limited to post-partum depression, depression affiliated with neurological diseases, major depression, seasonal affective disorder, depressive disorders, etc.), inflammation, Lyme disease, stroke, neurological diseases (such as Parkinson's and Alzheimer's), and gastrointestinal issues (including those in Parkinson's disease). The devices described herein can be used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. The devices described herein can be used for the treatment of inflammatory skin conditions. The neuromodulation devices, e.g., neurostimulation devices, described herein can be used for the treatment of chronic fatigue syndrome. The neuromodulation devices, e.g., neurostimulation devices described herein can be used for the treatment of chronic inflammatory symptoms and flare ups. Bradykinesia, dyskinesia, gait dysfunction, dystonia and/or rigidity may also be treated according to several embodiments. In several embodiments, rehabilitation as a result of certain events are treated, for example, rehabilitation from stroke or other cardiovascular events. In several embodiments, treatment of involuntary and/or repetitive movements is provided, including but not limited to tics, twitches, etc. (including, for example, Tourette Syndrome, tic disorders). Rhythmic and non-rhythmic involuntary movements may be controlled in several embodiments. Involuntary vocal tics and other vocalizations may also be treated. Systems and methods to reduce habituation and/or tolerance to stimulation in the disorders and symptoms identified herein are provided in several embodiments by, for example, introducing variability in stimulation parameter(s) described herein.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation), headache, back pain, joint pain and stiffness, muscle pain and tension, etc.

In several embodiments, the device 34 described herein can be used for the treatment of cardiac conditions (such as atrial fibrillation, hypertension, and stroke) and for the treatment of immune dysfunction. Epilepsy is treated in one embodiment. The devices described herein can be used to stimulate the to balance the autonomic nervous system. The devices described herein can be used sympathetic/parasympathetic nervous systems. Dysfunction or imbalance of the autonomic nervous system is believed to be a potential underlying mechanism for various chronic diseases. Autonomic dysfunction can develop when the nerves of the ANS are damaged or degraded or without any known neural pathology. This condition is called autonomic neuropathy or dysautonomia. Autonomic dysfunction can range from mild to life-threatening and can affect part of the ANS or the entire ANS. Sometimes the conditions that cause problems are temporary and reversible. Others are chronic, or long term, and may continue to worsen over time. Examples of chronic diseases that are associated with autonomic dysfunction include, but are not limited to, diabetes, Parkinson's disease, tremor, cardiac arrhythmias including atrial fibrillation, hypertension, overactive bladder, urinary incontinence, fecal incontinence, inflammatory bowel diseases, rheumatoid arthritis, migraine, depression, and anxiety.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc. Other disorders (e.g., hypertension, dexterity, and cardiac dysrhythmias) can also be treated using the embodiments described herein.

### Terminology

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the example term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps. However, some embodiments can consist or consist essentially of any number of stated elements or steps disclosed herein.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The methods disclosed herein include certain actions taken by a practitioner; however, they can also include any third-party instruction of those actions, either expressly or by implication. For example, actions such as “percutaneously stimulating an afferent peripheral nerve” includes “instructing the stimulation of an afferent peripheral nerve.”

What is claimed is:

1. A self-wetting electrode for transcutaneous electrical stimulation comprising:
a conductive backing layer; and
a skin contact layer disposed on the conductive backing layer, the skin contact layer configured to deliver electrical current from the conductive backing layer to the skin for transcutaneous electrical stimulation, the skin contact layer comprising an antistatic material configured to bloom to a skin facing surface of the skin contact layer to form a layer of conductive lubricant when the skin contact layer is in contact with a heat source.

2. The self-wetting electrode of claim 1, wherein the skin facing surface is not coated with a hydrogel or liquid.

3. The self-wetting electrode of claim 1, wherein the skin contact layer comprises silicone.

4. The self-wetting electrode of claim 3, wherein the antistatic material is incompatible with the silicone.

5. The self-wetting electrode of claim 1, wherein the skin contact layer comprises silver chloride.

6. The self-wetting electrode of claim 1, wherein the heat source is body heat of a user.

7. The self-wetting electrode of claim 1, wherein the antistatic material blooms to the skin facing surface of the skin contact layer to form the layer of conductive lubricant when the self-wetting electrode is placed on a desired location of a user's skin.

8. The self-wetting electrode of claim 1, wherein the antistatic material stops blooming to the skin facing surface after the layer of conductive lubricant covers substantially all the area of the skin facing surface.

9. A self-wetting electrode for transcutaneous electrical stimulation comprising:
a conductive backing layer; and
a skin contact layer disposed on the conductive backing layer, the skin contact layer configured to deliver electrical current from the conductive backing layer to the skin for transcutaneous electrical stimulation, the skin contact layer comprising an antistatic material configured to form a layer of conductive lubricant when the skin contact layer is in contact with a heat source.

10. The self-wetting electrode of any one of claims 1 to 9, wherein the layer of conductive lubricant is non-sticky and moisturizing.

11. The self-wetting electrode of any one of claims 1 to 9, wherein the layer of conductive lubricant is configured to provide a higher electrical conductivity between the skin contact layer and a skin location where the self-wetting electrode is placed on as compared to the electrical conductivity between the skin contact layer and the skin location without the layer of conductive lubricant.

12. The self-wetting electrode of any one of claims 1 to 9, wherein the layer of conductive lubricant is configured to provide adequate electrical conductivity between the skin contact layer and a skin location where the self-wetting electrode is placed on to comfortably deliver transcutaneous electrical stimulation.

13. The self-wetting electrode of any one of claims 1 to 9, wherein an amount of the antistatic material is sufficient to cover substantially all of the skin facing surface of the skin contact layer.

14. The self-wetting electrode of any one of claims 1 to 9, wherein an amount of the antistatic material is configured to not impact properties of the skin contact layer.

15. The self-wetting electrode of claim 14, wherein the properties of the skin contact layer comprise durability, conductivity, and shore hardness.

16. The self-wetting electrode of any one of claims 1 to 9, wherein the conductive backing layer comprises a metal foil.

17. The self-wetting electrode of any one of claims 1 to 9, wherein the skin contact layer comprises a polymer, plastic, or rubber material, and a conductive filler material dispersed substantially evenly throughout the polymer, plastic, or rubber material.

18. The self-wetting electrode of claim 17, wherein the conductive filler material comprises single wall carbon nanotubes.

19. The self-wetting electrode of claim 18, wherein a loading of single wall carbon nanotubes is between about 1% and about 5%.

20. The self-wetting electrode of claim 17, wherein the antistatic material is dispersed substantially evenly throughout the polymer, plastic, or rubber material.

21. The self-wetting electrode of any one of claims 1 to 9, wherein the skin contact layer comprises a thickness of between 0.5 mm and 10 mm.

22. The self-wetting electrode of any one of claims 1 to 9, wherein the skin contact layer has a Shore hardness between about 35 A to about 65 A.

23. A method of delivering transcutaneous electrical stimulation to a user, comprising:
providing a wearable device comprising at least one self-wetting electrode comprising a conductive backing layer, and a skin contact layer comprising an antistatic material configured to bloom to a skin facing surface of the skin contact layer to form a layer of conductive lubricant when the skin contact layer is in contact with skin of the user;
positioning the skin contact layer of the at least one self-wetting electrode on a desired location on the skin, wherein the skin facing surface of the skin contact layer is in direct contact with the skin;
waiting for the antistatic material to bloom to the skin facing surface of the skin contact layer; and
activating the wearable device, thereby delivering electrical current through the at least one self-wetting electrode to the desired location on the skin.

24. The method of claim 23, wherein the skin facing surface is not coated with a hydrogel or liquid.

25. The method of claim 23, wherein the conductive backing layer comprises a metal foil.

26. The method of claim 23, wherein the skin contact layer comprises a polymer, plastic, or rubber material, and a conductive filler material dispersed substantially evenly throughout the polymer, plastic, or rubber material.

27. The method of claim 23, wherein the skin contact layer comprises silver chloride.

28. The method of claim 23, wherein the conductive filler material comprises single wall carbon nanotubes.

29. The method of claim 28, wherein a loading of single wall carbon nanotubes is between about 1% and about 5%.

30. The method of any one of claims 23 to 28, wherein the antistatic material is dispersed substantially evenly throughout the polymer, plastic, or rubber material.

31. The method of any one of claims 23 to 28, wherein the skin contact layer comprises silicone.

32. The method of claim 31, wherein the antistatic material is incompatible with the silicone.

33. The method of any one of claims 23 to 28, wherein the conductive lubricant is non-sticky and moisturizing.

34. The method of any one of claims 23 to 28, wherein the antistatic material bloomed to the skin facing surface of the skin contact layer provides a higher electrical conductivity between the skin contact layer and the skin as compared to the electrical conductivity between the skin contact layer and the skin when there is no antistatic material bloomed to the skin facing surface.

35. The method of any one of claims 23 to 28, wherein an amount of the antistatic material is sufficient to cover substantially all of the skin facing surface of the skin contact layer.

36. The method of any one of claims 23 to 28, wherein an amount of the antistatic material is configured to not impact properties of the skin contact layer.

37. The method of claim 36, wherein the properties of the skin contact layer comprise durability, conductivity, and Shore hardness.

38. The method of any one of claims 23 to 28, wherein the skin contact layer comprises a thickness of between 0.5 mm and 10 mm.

39. The method of any one of claims 23 to 28, wherein the bloomed antistatic material covers substantially all of an area of the skin facing surface.

40. A wearable device for delivering transcutaneous electrical stimulation to a user, comprising:
a wearable band comprising a strap configured to be worn around a body part;
at least one electrode disposed on the strap;
a flexible circuit disposed within the strap;
a controller;
a power source; and
at least one reservoir configured to store wetting agent,
wherein the at least one electrode comprises a conductive backing layer and a skin contact layer disposed on the conductive backing layer and the skin contact layer is configured to deliver electrical current from the conductive backing layer to skin of the user for transcutaneous electrical stimulation,
wherein the at least one reservoir is in the wearable band and/or the at least one electrode, and
wherein the flexible circuit is in electrical communication with the at least one electrode, and the controller.

41. The wearable device of claim 40, wherein the wetting agent is delivered from the at least one reservoir to a skin facing surface of the skin contact by capillary action, osmosis, or micropump when the at least one electrode is placed on the skin of the user.

42. The wearable device of claim 40, wherein the wetting agent is water, electrolyte fluid or gel, salt solvent, or sweat of the user.

43. The wearable device of claim 40, wherein the at least one reservoir is configured to capture sweat of the user.

44. The wearable device of any one of claims 40 to 43, wherein the at least one reservoir comprises micro-sized structures.

45. The wearable device of claim 44, wherein the micro-sized structures comprise microchannels, grooves, and ridges.

46. The wearable device of any one of claims 40 to 43, wherein the at least one reservoir is configured to be pre-filled and/or re-filled by the user.

47. A method of wetting an electrode for transcutaneous electrical stimulation, comprising:
providing a wearable device comprising at least one electrode comprising a conductive backing layer and a skin contact layer comprising a skin facing surface;
positioning the at least one electrode on a desired location on skin of a user, wherein the skin facing surface of the skin contact layer is in direct contact with the skin;
activating the wearable device to perform a sweat induction session by applying a first electrical stimulus to at least the desired location so as to induce sweat between the skin facing surface of the skin contact layer and the skin; and
activating the wearable device to perform a tremor treatment session by applying a second electrical stimulus to at least the desired location through the induced sweat.

48. The method of claim 47, wherein the sweat induction session and the tremor treatment session are repeated for a plurality of cycles.

49. The method of claim 47, wherein the sweat induction session is about 1 minute and the tremor treatment session is about 40 minutes.

50. The method of claim 47, wherein the sweat induction session is configured to induce sufficient sweat to maintain sufficient conductivity between the at least one electrode and the skin.

51. The method of any one of claims 47 to 50, wherein the first electrical stimulus has an amplitude sufficiently high to activate C-type sudomotor neurons that are in target nerves and that innervate downstream (anterograde) sweat glands.

52. The method of any one of claims 47 to 50, wherein the first electrical stimulus has an amplitude between about 1 mA and about 10 mA.

53. The method of any one of claims 47 to 50, wherein the first electrical stimulus has a trapezoidal waveform configured to block the afferent pain signals caused by activating Aδ and C-type pain fibers.

54. The method of any one of claims 47 to 50, wherein the second electrical stimulus has a maximum amplitude different from a maximum amplitude of the first electrical stimulus.

55. The method of any one of claims 47 to 50, wherein the second electrical stimulus has an amplitude between about 1 to about 25 mA.

56. An in-line electrode system for delivering transcutaneous electrical stimulation to a target nerve of a limb, comprising:
two parallel counter electrodes; and
a stimulating electrode between and parallel to the two parallel counter electrodes,
wherein the stimulating electrode and the two counter electrodes are configured to be placed longitudinally along the target nerve on the same side of the limb relative to the target nerve.

57. The in-line electrode system of claim 56, wherein a first distance between the stimulating electrode and a first of the two counter electrodes and a second distance between the stimulating electrode and a second of the two counter electrodes are the same.

58. The in-line electrode system of claim 57, wherein the first and second distances are about 0.5 mm to about 5.0 mm.

59. A concentric electrode system for delivering transcutaneous electrical stimulation comprising:
a stimulating electrode; and
a counter electrode, wherein the stimulating electrode is configured to be placed around the counter electrode.

60. The concentric electrode system of claim 59, wherein a center of the counter electrode is coaxial with a center of the stimulating electrode.

61. The concentric electrode system of any one of claim 59 or 60, wherein a shape of the stimulating electrode is round or square.

62. The concentric electrode system of any one of claim 59 or 60, wherein an edge-to-edge gap between the counter electrode and the stimulating electrode is about 0.5 mm to about 5.0 mm.

63. An electrode system that reduces user discomfort caused by sweat inducing transcutaneous electrical stimulation, comprising:
an anode configured to be placed along a target nerve of the user at a distance from a hand of the user; and
a cathode positioned relative to the anode so as to be along the target nerve and between the anode and the hand.

64. The electrode system of claim 63, wherein the cathode is a negative pole and discharges anions.

65. The electrode system of claim 63, wherein the anode is a positive pole and discharges cations.

66. The electrode system of claim 63, wherein the sweat inducing transcutaneous electrical stimulation is delivered in a direction from the anode to the cathode.

67. The electrode system of claim 63, wherein the sweat inducing transcutaneous electrical stimulation is not delivered in a direction from the cathode to the anode.

68. The electrode system of any one of claims 63 to 67, wherein the sweat inducing transcutaneous electrical stimulation does not activate Aδ and C-type pain fibers in the user.

69. The electrode system of any one of claims 63 to 67, wherein the sweat inducing transcutaneous electrical stimulation has a waveform with a decay rate.

70. The electrode system of claim 69, wherein the waveform is a trapezoidal.

71. The electrode system of any one of claims 63 to 67, wherein the sweat inducing transcutaneous electrical stimulation is configured to block afferent pain signals caused by activating Aδ and C-type pain fibers.

72. The electrode system of any one of claims 63 to 67, wherein the cathode is distal of the anode.

73. The electrode system of any one of claims 63 to 67, wherein the cathode and the anode are on a same side of an arm of the user.

74. The electrode system of any one of claims 63 to 67, wherein the target nerve is a median nerve.

75. The electrode system of any one of claims 63 to 67, wherein the target nerve is a ulnar nerve.

76. The electrode system of any one of claims 63 to 67, wherein the target nerve is a radial nerve.

77. The use of any one of the devices, systems and methods of the preceding claims to reduce the dose of one or more drugs or pharmacological agents delivered to a patient, wherein the dose may for example include the amount, duration, number or frequency of the drug or pharmacological agent.

78. The use of any one of the devices, systems and methods of the preceding claims to enhance the efficacy of one or more drugs or pharmacological agents delivered to a patient.

79. The use of any one of the devices, systems and methods of the preceding claims for treatment of depression (including but not limited to post-partum depression, depression affiliated with neurological diseases, major depression, seasonal affective disorder, depressive disorders, etc.), inflammation (such as neuroinflammation), Lyme disease, stroke, neurological diseases (such as Parkinson's and Alzheimer's), and gastrointestinal issues (including those in Parkinson's disease).

80. The use of any one of the devices, systems and methods of the preceding claims for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases.

81. The use of any one of the devices, systems and methods of the preceding claims for the treatment of inflammatory skin conditions.

82. The use of any one of the devices, systems and methods of the preceding claims for the treatment of chronic fatigue syndrome.

83. The use of any one of the devices, systems and methods of the preceding claims for the treatment of chronic inflammatory symptoms and flare ups.

84. The use of any one of the devices, systems and methods of the preceding claims for the treatment of cardiac conditions (such as atrial fibrillation, hypertension, and stroke).

85. The use of any one of the devices, systems and methods of the preceding claims for the treatment of immune dysfunction.

86. The use of any one of the devices, systems and methods of the preceding claims to stimulate the autonomic nervous system.

87. The use of any one of the devices, systems and methods of the preceding claims to balance the sympathetic/parasympathetic nervous systems.

88. The use of any one of the devices, systems and methods of the preceding claims in a system and/or method which further comprises a wrist worn, leg worn or head (e.g., ear) worn device.

89. The use of any one of the devices, systems and methods of the preceding claims to reduce the dose of one or more drugs or pharmacological agents delivered to a patient, wherein the dose may for example include the amount, duration, number or frequency of the drug or pharmacological agent.

90. The use of any one of the devices, systems and methods of the preceding claims to enhance the efficacy of one or more drugs or pharmacological agents delivered to a patient.

91. The use of any one of the devices, systems and methods of the preceding claims for the treatment of pain.

92. The use of any one of the devices, systems and methods of the preceding claims for the treatment of pain whereby nerves are stimulated at a location different from the location of the pain.

93. The use of any one of the devices, systems and methods of the preceding claims for the treatment of pain whereby nerves are stimulated at a location different from the location of the pain, namely stimulation is provided at the wrist or other site on the arm and the pain to be treated is at or near the head or leg region.

94. The use of any one of the devices, systems and methods of the preceding claims to treat microbial infection and/or symptoms of microbial infection.

95. The use of any one of the devices, systems and methods of the preceding claims to treat autoimmune disorders and/or pain.

96. The use of any one of the devices, systems and methods of the preceding claims for the treatment of one or more of bradykinesia, dyskinesia, gait dysfunction, dystonia and/or rigidity.

97. The use of any one of the devices, systems and methods of the preceding claims for rehabilitation or physical therapy.

98. The use of any one of the devices, systems and methods of the preceding claims for the treatment of pain whereby nerves are stimulated at a location different from the location of the pain.

99. The use of any one of the devices, systems and methods of the preceding claims for the treatment of pain whereby nerves are stimulated at a location different from the location of the pain, namely stimulation is provided at the wrist or other site on the arm and the pain to be treated is at or near the head or leg region.

100. The use of any one of the devices, systems and methods of the preceding claims for
(a) stimulating one, two or all of the median nerve, radial nerve, and ulnar nerve and/or
(b) stimulating one or more of the auriculotemporal, great auricular, vagus, trigeminal or cranial nerves.

101. The use of any one of the devices, systems and methods of the preceding claims wherein the neuromodulation of the nerve affects neurotransmitter release, uptake and/or metabolism; increases neurotransmitter release, uptake and/or metabolism; decreases neurotransmitter release, uptake and/or metabolism; balances neurotransmitter release, uptake and/or metabolism by both increasing and decreasing neurotransmitter activity; activates or down regulates the dopaminergic system; activates or down regulates the serotonergic system; regulates the brain-gut axis; treats depression (including but not limited to post-partum depression, depression affiliated with neurological diseases, major depression, seasonal affective disorder, depressive disorders, etc.); treats inflammation (e.g., neuroinflammation); treats Lyme disease; treats stroke; treats neurological diseases (such as Parkinson's and Alzheimer's); treats gastrointestinal issues (including those in Parkinson's disease); treats inflammatory bowel disease (such as Crohn's disease, colitis, and functional dyspepsia), rheumatoid arthritis, multiple sclerosis, psoriatic arthritis, osteoarthritis, psoriasis and other inflammatory diseases; treats inflammatory skin conditions; treats chronic fatigue syndrome; treats chronic inflammatory symptoms and flare ups; treats cardiac conditions (such as atrial fibrillation, hypertension, and stroke); treats epilepsy; treats immune dysfunction; stimulates the autonomic nervous system; balance the sympathetic/parasympathetic nervous systems; treats habituation; treats mood disorders; treats pain (e.g., back pain, joint pain, stiffness, muscle pain, tension); treats migraine or other headache conditions; treats pain syndromes (e.g., trigeminal neuralgia, fibromyalgia, complex regional pain syndrome); treats microbial Infections (e.g., bacteria, viruses, fungi, and parasites); treats tetanus; treats meningitis; treats urinary tract infection; treats mononucleosis; treats autoimmune disorders; treats bradykinesia; treats dyskinesia; treats Gait dysfunction; treats dystonia; treats rigidity; treats hypertension; treats tinnitus; and/or treats dexterity.

102. The use of any one of the devices, systems and methods of the preceding claims to treat
(a) Tourette Syndrome and/or tic disorders, and/or associated symptoms, and/or
(b) rhythmic and/or non-rhythmic involuntary movements.

103. The use of any one of the devices, systems and methods of the preceding claims configured for placement on a limb, for example, an arm and/or a leg.

104. The use of any one of the devices, systems and methods of the preceding claims configured for placement on at least one limb to treat restless leg syndrome, periodic limb movement disorder, repetitive movements of the limbs and abnormal sensation.

105. The use of any one of the devices, systems and methods of the preceding claims configured for placement on at least one limb to treat one, two, three or more of the following nerves: peroneal, saphenous, tibial, femoral, and sural.

106. The use of any one of the devices, systems and methods of the preceding claims wherein the stimulation is provided in a burst pattern such as theta burst.

107. A method of delivering transcutaneous electrical stimulation to a user, comprising:
providing a wearable device comprising at least one self-wetting electrode comprising a conductive backing layer, and a skin contact layer comprising an antistatic material configured to bloom to a skin facing surface of the skin contact layer to form a layer of conductive lubricant when the skin contact layer is in contact with skin of the user;
positioning the skin contact layer of the at least one self-wetting electrode on a desired location on the skin, wherein the skin facing surface of the skin contact layer is in direct contact with the skin;
determine an impedance of the self-wetting electrode; and
activating the wearable device at least in part based on the impedance, thereby delivering electrical current through the at least one self-wetting electrode to the desired location on the skin.

108. The method of claim 107, wherein the impedance is based at least in part on a relative conductance between the peripheral nerve electrode and the skin of the user.
